(19) United States
(12) Patent Application Publication
(10) Pub. No.: US 2022/0226349 A1
(43) Pub. Date: Jul. 21, 2022

(54) TOPICAL NEUROSTEROID FORMULATIONS

(71) Applicant: Arizona Board of Regents on Behalf of the University of Arizona, Tucson, AZ (US)

(72) Inventors: Roberta Diaz Brinton, Tucson, AZ (US); Kathleen Rodgers, Tucson, AZ (US); Yu Jin Kim, Tucson, AZ (US); Heidi Mansour, Tucson, AZ (US)

(21) Appl. No.: 17/677,676

(22) Filed: Feb. 22, 2022

Related U.S. Application Data
(63) Continuation-in-part of application No. PCT/US2020/046905, filed on Aug. 19, 2020.
(60) Provisional application No. 62/888,826, filed on Aug. 19, 2019.

(51) Int. Cl. A61K 31/57; A61K 47/10; A61K 47/26; A61K 47/14; A61K 9/00; A61K 47/69

(52) U.S. Cl. CPC A61K 31/57; A61K 47/10; A61K 47/6951; A61K 47/14; A61K 9/0021; A61K 47/26

(57) ABSTRACT

Formulations for treating or preventing neuronal damage and/or the associated cognitive decline or impairment, caused by Alzheimer's disease and/or other neurodegenerative diseases, contain a therapeutic agent and a pharmaceutically acceptable carrier, wherein the therapeutic agent is dissolved in the pharmaceutically acceptable carrier. The formulations provide a safe, stable, convenient way to store and deliver high concentrations of the therapeutic agent, particularly when the therapeutic agent is lipophilic. The therapeutic agent can be a neurosteroid, a derivative or analogue thereof, or a pharmaceutically acceptable salt of the neurosteroid or its derivative or analogue. The pharmaceutically acceptable carrier can contain water, one or more lipophilic compounds, a surfactant, and optionally a co-surfactant. Generally, the carrier forms a stable microemulsion.

### CROSS REFERENCE TO RELATED APPLICATION

This application is a Continuation-in-Part of PCT/US2020/046905 filed Aug. 19, 2020, entitled “TOPICAL NEUROSTEROID FORMULATIONS”, by Roberta Diaz Brinton, Kathleen Rodgers, Yu Jin Kim, and Heidi Mansour, under 35 U.S.C. § 371, wherein PCT/US2020/046905 claims priority to and benefit of U.S. Provisional Application 62/888,826 filed Aug. 19, 2019, both of which are herein incorporated by reference in their entirety.

### FIELD OF THE INVENTION

This invention is in the field of pharmaceutical compositions for preventing and reversing neurological deficits associated with Alzheimer's disease and/or other neurodegenerative diseases, and methods of use thereof, particularly formulations containing 3a-hydroxy-5a-pregnan-20-one or its derivatives and analogues.

### BACKGROUND OF THE INVENTION

Alzheimer's disease (AD) is a progressive multifactorial disease, affecting more than 50 million people worldwide, and will reach 75 million in 2030 and 131.5 million in 2050 Alzheimer's is the most common dementia of late-life. The mean incidence of AD is 1-3% and is associated with an overall prevalence of 10-30% in persons over 65 years of age which, globally, is predicted to nearly double every 20 years. On average, persons will live with Alzheimer's disease for 10 years. In the United States, total costs for caring for the 5 million persons living with the disease is estimated at S200 billion and are projected to rise to S1.1 trillion by 2050. To date, no interventions have demonstrated therapeutic efficacy to prevent, delay or treat AD and several have accelerated disease progression (http://www.alzforum.org/therapeutics).

Administration of neurotrophic factors, such as nerve growth factor and insulin-like growth factor, have been suggested to stimulate neuronal growth within the central nervous system. However, in spite of significant efforts, to date no satisfactory therapeutic compositions or treatment methods exists to repair, or counteract, the neuronal damage and/or the associated cognitive decline or impairment, caused by Alzheimer's disease.

3α-hydroxy-5α-pregnan-20-one (allopregnanolone) has now been demonstrated in animal studies and a phase I human trial to have a positive impact in limiting or even remediating memory loss in some Alzheimer's patients. This trial used intravenous administration, which is of limited general applicability. Other types of formulations for treatment of patients with impaired cognition have been developed and tested in animals and on humans. See, for example, PCT/US2019/022056 and US20100204192.

It is now known that the timing of administration is critical. This is a problem when the patient population is mentally deficient, and administration of the treatment dependent on busy caretakers who may be dealing with poor patient compliance.

There is a need for new treatment modalities directed to improving the adverse neurological conditions associated with Alzheimer's disease and/or other neurodegenerative diseases.

It is an object of the invention to provide compositions and methods for the treatment or prevention of neuronal damage and/or the associated cognitive decline or impairment, caused by Alzheimer's disease and/or other neurodegenerative diseases.

### BRIEF SUMMARY OF THE INVENTION

Formulations for treating or preventing neuronal damage and/or the associated cognitive decline or impairment, caused by Alzheimer's disease and/or other neurodegenerative diseases, contain a therapeutic agent dissolved in a pharmaceutically acceptable carrier for topical administration or in a microneedle transdermal patch. The formulations provide a safe, stable, convenient way to store and deliver high concentrations of the therapeutic agent, particularly when the therapeutic agent is lipophilic.

The therapeutic agent is preferably a neurosteroid, a derivative or analogue thereof, or a pharmaceutically acceptable salt of the neurosteroid or its derivative or analogue. In the most preferred embodiments, the therapeutic agent is 3a-hydroxy-5a-pregnan-20-one (allopregnanolone), a derivative or analogue thereof, or a pharmaceutically acceptable salt of the derivative or analogue.

In one embodiment, the carrier is a microemulsion formed of water, one or more lipophilic compounds, a solubilizer, a surfactant, and optionally a co-surfactant. In some embodiments, the solubility of the therapeutic agent in the carrier is at least about 6-fold, at least about 10-fold, at least about 14-fold, at least about 18-fold, at least about 22-fold, or at least about 26-fold higher than the solubility of the therapeutic agent in a corresponding carrier without the one or more lipophilic compounds, surfactant, or co-surfactant, for example, as compared to an allopregnanolone solution for intravenous and intramuscular administration (1.5 mg/ml in 0.9% sodium chloride with 6% sulfobutyl-ether-beta-cyclodextrin solution) in phase 1 clinical trials

The one or more lipophilic compounds from the carrier can be selected from fatty acids, fatty acid esters, and combinations thereof. In some embodiments, the lipophilic compounds are C₆-C₁₂ medium-chain, saturated or non-saturated, mono-, di- or tri-glycerides, such as caprylic monoglyceride, caprylic diglyceride, capric monoglyceride, capric diglyceride, and combinations thereof. In some embodiments, the carrier contains an oil, which encompasses the lipophilic compounds. An exemplary oil is CAPMUL® MCM.

The surfactant from the carrier can be a non-ionic surfactant, such as polysorbates, sorbitan alkanoates, polyoxyethylene fatty acid esters, and combinations thereof. In some embodiments, the surfactant is sorbitan monooleate or Polysorbate 80. In other embodiments, the surfactant is a combination of sorbitan monooleate and Polysorbate 80, optionally at a weight ratio of about 1. The co-surfactant from the carrier can be a short-chain (e.g., C₂-C₅), medium-chain (e.g., C₆-C₁₂), or long-chain (e.g., C₁₃-C₂₁) alcohol or amine. In some embodiments, the co-surfactant is diethylene glycol monoethyl ether.

The carrier can also contain a transdermal penetration enhancer, such as ethanol, propylene glycol, or glycerol. In some embodiments, the transdermal penetration enhancer is ethanol.

The concentrations of the components of the formulations can vary. For example, the concentration of the therapeutic agent in the formulations can be between about 0.5 and about 100 mg/ml, preferably between 6 and 50, most preferably between 6 and 39 mg/ml. The weight percent of the one or more lipophilic compounds or the oil relative to the carrier can be more than 0.01% and up to 30%, preferably between about 2% and about 15%, most preferably between 2 and 7%. The weight percent of the surfactant or the combination of the surfactant and the co-surfactant relative to the carrier can be between about 10% and about 90%, preferably between about 60% and about 90%, most preferably between about 73 and 88%. In the preferred embodiment, the weight percent of the transdermal penetration enhancer relative to the carrier is up to about 20%. The weight percent of water relative to the carrier can be more than 1% and up to about 90%, preferably between about 4% and about 20% and 90%, and most preferably, between about 57% and about 88%.

Exemplary formulations contain the therapeutic agent dissolved in a carrier containing water, one or more lipophilic compounds, a surfactant, a co-surfactant, and a tissue penetration enhancer. In some embodiments, the therapeutic agent is 3a-hydroxy-5a-pregnan-20-one; the one or more lipophilic compounds are selected from caprylic monoglyceride, caprylic diglyceride, capric monoglyceride, capric diglyceride, and combinations thereof; the surfactant is sorbitan monooleate, Polysorbate 80, or a combination of sorbitan monooleate and Polysorbate 80 at a weight ratio of about 1; the co-surfactant is diethylene glycol monoethyl ether; and the transdermal penetration enhancer is ethanol. Optionally, the carrier contains CAPMUL® MCM, wherein the one or more lipophilic compounds originally belonged to the CAPMUL® MCM.

In some forms, the formulation is administered using a microneedle device, such as a microneedle patch, to a subject in need thereof. Exemplary microneedle devices include at least two components: a plurality of microneedles and a substrate to which the base of the microneedles is secured or integrated. In some forms, the microneedles are biodegradable and contain the formulation.

Dosage unit kits for treating or preventing neuronal damage and/or the associated cognitive decline or impairment, caused by Alzheimer's disease and/or other neurodegenerative diseases, contain a formulation disclosed herein. In some embodiments, the kits have one or more containers for dry components and one or more containers for liquid components, which are mixed together to form the formulation before administration to a subject in need thereof.

Methods for treating or preventing neuronal damage and/or the associated cognitive decline or impairment, caused by Alzheimer's disease and/or other neurodegenerative diseases, generally include administering an effective amount of a formulation disclosed herein to a subject in need thereof. The formulation can be administered transdermally or transcutaneously. In some embodiments, the formulation is administered using microneedles, intranasal spray, buccal film, transdermal patch, or sublingual tablet or spray.

### BRIEF DESCRIPTION OF THE DRAWINGS

FIGS. 1A-1C are pseudo ternary phase diagrams of different oil compositions. FIG. 1A: CAPMUL® MCM, [surfactant (SPAN® 80):co-surfactant (TRANSCUTOL® P), 1:1, w/w], and water; FIG. 1B: CAPMUL® MCM, [surfactant (TWEEN® 80):co-surfactant (TRANSCUTOL® P), 1:1, w/w], and water; and FIG. 1C: CAPMUL® MCM, [surfactants (TWEEN® 80 and SPAN® 80):co-surfactant (TRANSCUTOL® P), 1:1:8, w/w/w], and water. The shaded regions indicate stable and mono-phase microemulsions (MEs).

FIGS. 2A-2C are bar graphs showing the in vitro cell viability of HaCaT (human skin, FIG. 2A), RPMI 2650 (human nasal, FIG. 2B), and TR 146 (human buccal, FIG. 2C) cells after treatment of allopregnanolone. The control group was treated with the corresponding cell culture medium containing 1% ethanol, without allopregnanolone. Each viability value represents mean±SD, n=6.

FIG. 3 is a bar graph showing the saturated solubilities of allopregnanolone in the MEs. Each solubility value represents mean±SD (n=3).

FIG. 4 is a graph showing the in vitro cumulative permeation amount of allopregnanolone (μg/cm²) over time (h) from ME formulations with or without penetration enhancers. The effect of penetration enhancers on the in vitro permeation profiles of allopregnanolone was determined using the STRAT-M® membrane for 48 h at 32° C. Each data point represents mean±SD (n=3-5). The composition of the three ME formulations is shown in Table 8.

FIG. 5 is a graph showing the in vitro cumulative permeation amount of allopregnanolone (μg/cm²) over time (h) from three ME formulations, i.e., ME-A, ME-B, and ME-C. The experiment was performed using the STRAT-M® membrane for 48 h at 32° C. Each data point represents mean±SD (n=3).

FIG. 6 is a graph showing the in vitro cumulative release percent of allopregnanolone (%) over time (h) from different ME formulations, i.e., ME-A, ME-B, and ME-C. The experiment was performed using the STRAT-M® membrane for 48 h at 32° C. Each data point represents mean±SD (n=3)

FIGS. 7A-7C are cross-sectional views of exemplary microneedle devices. They correspond to FIGS. 1A-1C of U.S. Pat. No. 6,611,707. The devices in FIGS. 7A-7C each include a reservoir and are suitable for transdermal delivery of the formulations. The devices in FIGS. 7B and 7C include a deformable reservoir, wherein delivery is activated by manual, e.g., finger or thumb, pressure applied to compress the reservoir directly (7B) or indirectly (7C).

FIG. 8 is a cross-sectional view of another exemplary microneedle device. It corresponds to FIG. 2 of U.S. Pat. No. 6,611,707. Delivery of the formulation is activated by manual pressure applied via a plunger to compress the reservoir.

FIG. 9 is a cross-sectional view of another exemplary microneedle device. It corresponds to FIG. 3 of U.S. Pat. No. 6,611,707. Delivery of the formulation is activated by releasing a compressed spring which forces the plunger to compress the reservoir.

FIGS. 10A and 10B are cross-sectional views of exemplary microneedle devices having a multi-chamber reservoir. They correspond to FIG. 4A and 4B of U.S. Pat. No. 6,611,707.

FIG. 11 is a cross-sectional view of an exemplary microneedle device, which incorporates an osmotic pump to force the formulation out from the reservoir. It corresponds to FIG. 5 of U.S. Pat. No. 6,611,707.

### DETAILED DESCRIPTION OF THE INVENTION

### I. Definitions

Use of the term “about” is intended to describe values either above or below the stated value in a range of approximately +/−10.

Unless otherwise specified, the phrases “by weigh percent” and “by wt %,” refer to percent weight by weight, i.e. , % w/w.

The term “derivative” refers to compounds which are formed from a parent compound by one or more chemical reaction(s) but having a similar function. The term “analogue” refers to a chemical compound with a structure similar to that of another (reference compound) but differing from it in respect to a particular component, functional group, atom, etc., while retaining a similar function. The differences between the derivatives/analogues and their parent/reference compounds include, but are not limited to, replacement of one or more functional groups with one or more different functional groups, introducing or removing one or more substituents of the hydrogen atoms, converting an acid or base compound to its salt form or vice versa.

The term “pharmaceutically acceptable” refers to those compounds, materials, compositions, and/or formulations which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the United States Food and Drug Administration.

“Pharmaceutically acceptable salt” refers to the modification of the original compound by making the acid or base salts thereof. Examples of pharmaceutically acceptable salts include, but are not limited to, mineral or organic acid salts of basic residues such as amines and alkali or organic salts of acidic residues such as carboxylic acids. For original compounds containing a basic residue, pharmaceutically acceptable salts can be prepared by treating the compounds with an appropriate amount of a non-toxic inorganic or organic acid. Suitable inorganic acids include hydrochloric, hydrobromic, sulfuric, sulfamic, phosphoric, and nitric acids; suitable organic acids include acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic acids. For original compounds containing an acidic residue, pharmaceutically acceptable salts can be prepared by treating the compounds with an appropriate amount of a non-toxic base. Suitable non-toxic bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Generally, pharmaceutically acceptable salts can be prepared by reacting the free acid or base form of the original compounds with a stoichiometric amount of the appropriate base or acid, respectively, in water or in an organic solvent, or in a mixture thereof. Non-aqueous media like ether, ethyl acetate, ethanol, isopropanol, acetonitrile, or combinations thereof can be used. Lists of suitable pharmaceutically acceptable salts can be found in Remington's Pharmaceutical Sciences, 20th Ed., Lippincott Williams & Wilkins, Baltimore, Md., 2000, p. 704; and Handbook of Pharmaceutical Salts: Properties, Selection, and Use, Stahl and Wermuth, Eds., Wiley-VCH, Weinheim, 2002.

The term “neurosteroid” refers to endogenous or exogenous steroids that can alter neuronal excitability through interaction with ligand-gated ion channels and other cell surface receptors. In addition to their actions on neuronal membrane receptors, some of these steroids may also exert effects on gene expression via nuclear steroid hormone receptors.

Lipophilicity refers to the ability of a chemical compound to dissolve in fats, oils, lipids, and non-polar solvents such as hexane or toluene. Lipophilic substances tend to dissolve in other lipophilic substances. Lipophilic substances interact with themselves and with other substances through the London dispersion force. They have little to no capacity to form hydrogen bonds. When a molecule of a lipophilic substance is enveloped by water, surrounding water molecules enter into an “ice-like” structure over the greater part of its molecular surface, the thermodynamically unfavorable event that drives the lipophilic substance out of water. Generally, lipophilic substances are water insoluble. They have large partition coefficients, such as with a log Pow larger than 0.5, larger than 1, larger than 2, larger than 3, larger than 4, or larger than 5.

The term “microemulsion” refers to clear, thermodynamically stable, isotropic liquid mixtures of water (forming the aqueous phase), one or more lipophilic compounds (forming the oil phase), and surfactant, optionally in combination with co-surfactant. The aqueous phase may contain salt, buffering agent, and/or other ingredients. Optionally, the microemulsions can be formed of water, oil, surfactant, and optionally co-surfactant, wherein the one or more lipophilic compounds originally belonged to the oil before forming the microemulsions. In contrast to ordinary emulsions, microemulsions generally form upon simple mixing of the components and do not require the high shear conditions used in the formation of ordinary emulsions. The three basic types of microemulsions are direct (the oil phase dispersed in the aqueous phase, o/w), reversed (the aqueous phase dispersed in the oil phase, w/o), and bicontinuous. The surfactant molecules can form a monolayer at the interface between the oil phase and the aqueous phase, with the hydrophobic tails of the surfactant molecules dissolved in the oil phase and the hydrophilic head groups in the aqueous phase. In microemulsions, hydrophilic agents are typically incorporated by solubilization in the aqueous phase, whereas lipophilic agents are typically solubilized in the oil phase.

The term “oil” refers to natural or synthetic chemical substances that are lipophilic and not miscible with water. In some forms, an oil can be composed of a single lipophilic compound. In some forms, an oil can be a mixture containing different lipophilic compounds.

The term “surfactant” refers to amphiphilic compounds generally recognized in the art as having surface active qualities. Surfactants can be anionic, cationic, nonionic, and zwitterionic compounds. Generally, surfactants absorb to an interface between two immiscible phases, such as the interface between an aqueous phase and an oil phase.

The term “co-surfactant” refers to chemicals added to a process to enhance the effectiveness of a surfactant. Like surfactants, co-surfactants are amphiphilic that has an affinity for oil and aqueous phases. It is incorporated into the microemulsion systems to further decrease surface tension and introduce flexibility into the interfacial surfactant in the systems. Non-ionic surfactants (e.g., Tweens, Cremophor, Transcutol, Brij, Labrafil, TPGS, Gelucire, Solutol, Poloxamers, Spans, and Labrasol), lecithin, alcohols, alkanoic acids, alkanediols, and alkyl amines can function as co-surfactants in the microemulsion systems (Lawrence and Rees, Adv Drug Deliv Rev, 2000, 45:89-121; and Callender et al., Int J Pharm, 2017, 526:425-442). Exemplary co-surfactants include short-chain (e.g., C₂-C₅), medium-chain (e.g., C₆-C₁₂), and long-chain (e.g., C₁₃-C₂₁) alcohols or amines Co-surfactants can be used to increase the lipid-solubilizing capacity of microemulsion systems. Surfactants often organize well at a liquid/liquid boundary, which leads to relatively stiff interfaces or even liquid-crystal phases. To achieve ultralow interfacial tension for the microemulsion systems, a co-surfactant can be added to disturb this organization at the liquid/liquid interface. Co-surfactants can also be used to fine-tune the formulation phase behavior, for example, by expanding the temperature or salinity range of microemulsion formations.

The term “transdermal” refers to delivery across or into the epidermis, dermis, or both. Transdermal delivery can be achieved by using a transdermal penetration enhancer to decrease the barrier resistance. Transdermal delivery can be also achieved using a delivery device, such as a microneedle device, that can penetrate the epidermis, dermis, or both.

The term “transcutaneous” refers to penetrating, entering, or passing through the intact skin. This term is in contrast to the term “percutaneous,” which means through a disruption in the skin.

The term “transdermal penetration enhancer” refers to chemical agents which can penetrate into skin to reversibly decrease the barrier resistance, thereby improving transdermal drug delivery. There are many potential sites and modes of action for transdermal penetration enhancers. For example, the transdermal penetration enhancers may disrupt the packing motif in the intercellular lipid matrix. Alternatively, the transdermal penetration enhancers may increase drug partitioning into the tissue by acting as a solvent for the permeant within the membrane. Alternatively, the transdermal penetration enhancers may act on desmosomal connections between corneocytes or alter metabolic activity within the skin, or exerting an influence on the thermodynamic activity/solubility of the drug in its carrier.

The term “room temperature” refers to a temperature between 20-25° C., typically about 25° C.

The term “in need of treatment” as used herein refers to a judgment made by a caregiver (e.g., physician, nurse, nurse practitioner, or caregiver) that a subject requires or will benefit from treatment. This judgment is made based on a variety of factors that are in the realm of a caregiver's expertise, but that include the knowledge that the subject is ill, or will be ill, as the result of a condition that is treatable by the compositions disclosed herein.

The terms “treatment” and “treating” refer to the medical management of a subject with the intent to cure, ameliorate, stabilize, or prevent one or more symptoms of a disease, pathological condition, or disorder. This term includes active treatment toward the improvement of a disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “preventing” refers to administering a pharmaceutical composition prior to the onset or exacerbation of clinical symptoms or of a disease, pathological condition, or disorder so as to prevent a physical manifestation of aberrations associated with the disease, pathological condition, or disorder.

The term “effective amount” of a composition refers to a nontoxic but sufficient amount of the composition to provide the desired result. The exact amount required will vary depending on the severity of neural deterioration or neural loss caused by a neurological disease, neurological injury, and/or age-related neuronal decline or impairment.

### II. Compositions

Formulations and devices such as transdermal microneedle devices for treating or preventing the neuronal damage and/or the associated cognitive decline or impairment caused by Alzheimer's disease and/or other neurodegenerative diseases, generally contain a therapeutic agent and a pharmaceutically acceptable carrier, wherein the therapeutic agent is dissolved in the pharmaceutically acceptable carrier. The formulations provide a safe, stable, convenient way to store and deliver high concentrations of the therapeutic agent, particularly when the therapeutic agent is lipophilic.

The therapeutic agent is a neurosteroid. In the preferred embodiment, the therapeutic agent is 3a-hydroxy-5a-pregnan-20-one, a derivative or analogue thereof, or a pharmaceutically acceptable salt of the derivative or analogue.

The pharmaceutically acceptable carrier can contain water, one or more lipophilic compounds, a surfactant, and optionally a co-surfactant. Generally, the carrier forms a stable microemulsion. In some embodiments, the solubility of the therapeutic agent in the carrier is at least about 6-fold, at least about 10-fold, at least about 14-fold, at least about 18-fold, at least about 22-fold, or at least about 26-fold higher than the solubility of the allopregnanolone solution for intravenous and intramuscular administration (1.5 mg/ml in 0.9% sodium chloride with 6% sulfobutyl-ether-beta-cyclodextrin solution) in phase 1 clinical trials.

In some embodiments, the carrier also contains a transdermal penetration enhancer, such as diethylene glycol monoethyl ether. Preferably, the one or more lipophilic compounds, surfactant, co-surfactant, and/or transdermal penetration enhancer meets the requirements of the United States Food and Drug Administration as generally recognized as safe (GRAS) compounds.

A. Therapeutic Agents

The formulations contain a therapeutic agent. In some embodiments, the therapeutic agent is lipophilic, e.g., having a large partition coefficient such as with a log Pow larger than 0.5, larger than 1, larger than 2, larger than 3, larger than 4, or larger than 5 (“log Pow” is the partition coefficient of the agent in a biphasic system of octanol (“O”) and water (“w”)). The therapeutic agent is a neurosteroid, a derivative or analogue thereof, a pharmaceutically acceptable salt of the neurosteroid or the derivative or analogue, precursor or metabolites of the neurosteroid from its metabolic pathway. A large body of literature explores the potential for neurosteroid-based interventions of Alzheimer's disease, for example, Schneider et al., Arch Neurol, 2011, 68:58-66; Carlson et al., Alzheimers Dement, 2011, 7:396-401; Sperling et al., Lancet Neurol, 2012, 11:241-9; Brinton, Nat Rev Endocrinol, 2013, 9:241-50; Chen et al., PLoS One, 2011, 6:e24293; Singh et al., Neurobiol Aging, 2012, 33(8):1493-506; Wang et al., Proc Natl Acad Sci USA, 2010, 107:6498-503; Wang et al., J Neurosci, 2005, 25: 7986-92; Sun et al., Curr Alzheimer Res, 2012, 9:473-80; Lan et al., Hormones and behavior, 1994, 28:537-44; Reddy et al., Neurotherapeutics, 2009, 6:392-401; Simon et al., J Natl Cancer Inst, 1997, 89:1138-47; Irwin et al., Front Endocrinol (Lausanne), 2011, 2:117; Petersen, Nature Reviews Drug Discovery, 2003, 2:646-53; McKhann et al., Alzheimers Dement, 2011, 7:263-9; Green et al., JAMA, 2009, 302:2557-64; Collie et al., Psychopharmacol, 2006, 21:481-8; Falleti et al., J Clin Exp Neuropsychol, 2006, 28:1095-112; Lim et al., J Clin Exp Neuropsychol, 2012, 34:345-58; Bond et al., Psychol Med, 1974, 4:374-80; Sperling et al., Alzheimers Dementia, 2011, 7:367-85; Salloway et al., Neurology, 2009, 73:2061-70; and Weiner et al., Alzheimers Dement, 2012, 8:S1-68.

Exemplary neurosteroids include inhibitory neurosteroids which exert inhibitory actions on neurotransmission (e.g., tetrahydrodeoxycorticosterone, 3α-androstanediol, cholesterol, pregnanolone, and allopregnanolone); excitatory neurosteroids which have excitatory effects on neurotransmission (e.g., pregnenolone sulfate, epipregnanolone, isopregnanolone, dehydroepiandrosterone, dehydroepiandrosterone sulfate, and 24(S)-hydroxycholesterol); pheromones which can influence brain activity (e.g., androstadienol, androstadienone, androstenol, androstenone, and estratetraenol); and other neurosteroids such as progesterone, estradiol, and corticosterone.

In the preferred embodiment, the therapeutic agent is 3α-hydroxy-5α-pregnan-20-one (allopregnanolone, abbreviated as Allo, also known as brexanolone), a derivative or analogue thereof, or a pharmaceutically acceptable salt of the derivative or analogue. 3α-hydroxy-5α-pregnan-20-one is a naturally occurring metabolite of progesterone. It is produced in the central nervous system and was previously found to be an allosteric modulator of GABA receptors. Suitable derivatives or analogues of 3α-hydroxy-5α-pregnan-20-one include progesterone-like molecules that are natural precursors or metabolites of progesterone or synthetic variants of progesterone that exhibit equivalent neurogenic activity as 3α-hydroxy-5α-pregnan-20-one. Equivalent neuro-enhancing activity is defined as between about 30% and about 500%, between about 50% and about 300%, or between about 80% and about 200% of the neuro-enhancing activity of 3α-hydroxy-5α-pregnan-20-one.

In certain embodiments, the therapeutic agent is a substituted derivative of 3α-hydroxy-5α-pregnan-20-one, wherein one or more functional groups and/or hydrogen atoms of 3α-hydroxy-5α-pregnan-20-one are substituted. The substituents of the functional groups and/or hydrogen atoms include, but are not limited to:
a halogen atom, an alkyl group, a heteroalkyl group, an alkenyl group, a heteroalkenyl group, an alkynyl group, a heteroalkynyl group, an aryl group, a heteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{G1})(OR^{G2}), —OP(═O)(OR^{G1})(OR^{G2}), —BR^{G1}(OR^{G2}), —B(OR^{G1})(OR^{G2}), or —GR^{G1} in which —G is —O—, —S—, —NR^{G2}—, —C(═O)—, —S(═O)—, —SO₂—, —C(═O)O—, —C(═O)NR^{G2}—, —OC(′O)—, —NR^{G2}C(═O)—, —OC(═O)O—, —OC(═O)NR^{G2}—, —NR^{G2}C(═O)O—, —NR^{G2}C(═O)NR^{G3}—, —C(═S)—, —C(═S)S—, —SC(═S)—, —SC(═S)S—, —C(═NR^{G2})—, —C(═NR^{G2})O—, —C(═NRG²)NRG³—, —OC(═NRG²)—, —NR^{G2}C(═NR^{G3})—, —NR^{G2}SO₂—, —C(═NRG²)NRG³—, —OC(═NRG²)—, —NR^{G2}C(═NR^{G3})—, —NR^{G2}SO₂—, —NRG²SO₂NR^{G3}—, —NR^{G2}C(═S)—, —SC(═S)NR^{G2}—, —NR^{G2}C(═S)S—, —NRG²C(═S)NRG³—, —SO(═NRG²)—, —C(═S)NRG²—, —OC(═S)NRG²—, —NRG²C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O—, —SO₂NR^{G2}—, —BR^{G2}—, or —PR^{G2}—,
wherein each occurrence of R^{G1}, R^{G2},and R^{G3} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In certain embodiments, the hydrogen atom of the 3α carbon of 3α-hydroxy-5α-pregnan-20-one can be substituted as described above. Exemplary substituted derivatives include those described in Hawkinson et al., J. Pharmacology & Experimental Therapeutics, 287:198-207 (1998).

In certain embodiments, the hydrogen atom in the 3α-hydroxyl group can be substituted as described above. Exemplary substituted derivatives include 3α-ester derivatives and 3α-ether derivatives. The ester or ether group may contain an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group.

In certain embodiments, the 3α-hydroxyl group can be substituted as described above. Exemplary substituents of 3α-hydroxyl group include, but are not limited to, an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, and an optionally substituted heteroaryl group.

In certain embodiments, the 3α-hydroxyl group is replaced by an oxidized form of the hydroxyl group, such as a carboxylate group, an aldehyde group, or a carbonyl group.

As used herein, the alkyl group can be linear, branched, or cyclic. It is understood that a branched alkyl or a cyclic alkyl contains at least four and three carbon atoms, respectively. Optionally, the alkyl group can have 1-30 carbon atoms, i.e., C₁-C₃₀ alkyl. In some forms, the C₁-C₃₀ alkyl can be a linear C₁-C₃₀ alkyl, a branched C₄-C₃₀ alkyl, a cyclic C₃-C₃₀ alkyl, a linear or branched C₁-C₃₀ alkyl, a linear or cyclic C₁-C₃₀ alkyl, a branched or cyclic C₃-C₃₀ alkyl, or a linear, branched, or cyclic C₁-C₃₀ alkyl. Optionally, the alkyl group have 1-20 carbon atoms, i.e., C₁-C₂₀ alkyl. In some forms, the C₁-C₂₀ alkyl can be a linear C₁-C₂₀ alkyl, a branched C₄-C₂₀ alkyl, a cyclic C₃-C₂₀ alkyl, a linear or branched C₁-C₂₀ alkyl, a linear or cyclic C₁-C₂₀ alkyl, a branched or cyclic C₃-C₂₀ alkyl, or a linear, branched, or cyclic C₁-C₂₀ alkyl. Optionally, the alkyl group can have 1-10 carbon atoms, i.e., C₁-C₁₀ alkyl. In some forms, the C₁-C₁₀ alkyl can be a linear C₁-C₁₀ alkyl, a branched C₄-C₁₀ alkyl, a cyclic C₃-C₁₀ alkyl, a linear or branched C₁-C₁₀ alkyl, a linear or cyclic C₁-C₁₀ alkyl, a branched or cyclic C₃-C₁₀ alkyl, or a linear, branched, or cyclic C₁-C₁₀ alkyl.

The heteroalkyl group can be linear, branched, or cyclic. It is understood that a branched heteroalkyl or a cyclic heteroalkyl contains at least three and two carbon atoms, respectively, in addition to at least one heteroatom. Optionally, the heteroalkyl group can have 1-30 carbon atoms, i.e., C₁-C₃₀ heteroalkyl. In some forms, the C₁-C₃₀ heteroalkyl can be a linear C₁-C₃₀ heteroalkyl, a branched C₃-C₃₀ heteroalkyl, a cyclic C₂-C₃o heteroalkyl, a linear or branched C₁-C₃₀ heteroalkyl, a linear or cyclic C₁-C₃₀ heteroalkyl, a branched or cyclic C₂-C₃₀ heteroalkyl, or a linear, branched, or cyclic C₁-C₃₀ heteroalkyl. Optionally, the heteroalkyl group have 1-20 carbon atoms, i.e., C₁-C₂₀ heteroalkyl. In some forms, the C₁-C₀ heteroalkyl can be a linear C₁-C₂₀ heteroalkyl, a branched C₃-C₂₀ heteroalkyl, a cyclic C₂-C₂₀ heteroalkyl, a linear or branched C₁-C₂₀ heteroalkyl, a linear or cyclic C₁-C₂₀ heteroalkyl, a branched or cyclic C₂-C₂₀ heteroalkyl, or a linear, branched, or cyclic C₁-C₂₀ heteroalkyl. Optionally, the heteroalkyl group can have 1-10 carbon atoms, i.e., C₁-C₁₀ heteroalkyl. In some forms, the C₁-C₁₀ heteroalkyl can be a linear C₁-C₁₀ heteroalkyl, a branched C₃-C₁₀ heteroalkyl, a cyclic C₂-C₁₀ heteroalkyl, a linear or branched C₁-C₁₀ heteroalkyl, a linear or cyclic C₁-C₁₀ heteroalkyl, a branched or cyclic C₂-C₁₀ heteroalkyl, or a linear, branched, or cyclic C₁-C₁₀ heteroalkyl.

The alkenyl group can be linear, branched, or cyclic. It is understood that a branched alkenyl or a cyclic alkenyl contains at least four and three carbon atoms, respectively. Optionally, the alkenyl group can have 2-30 carbon atoms, i.e., C₂-C₃₀ alkenyl. In some forms, the C₂-C₃₀ alkenyl can be a linear C₂-C₃₀ alkenyl, a branched C₄-C₃₀ alkenyl, a cyclic C₃-C₃₀ alkenyl, a linear or branched C₂-C₃₀ alkenyl, a linear or cyclic C₂-C₃₀ alkenyl, a branched or cyclic C₃-C₃₀ alkenyl, or a linear, branched, or cyclic C₂-C₃₀ alkenyl. Optionally, the alkenyl group can have 2-20 carbon atoms, i.e., C₂-C₂₀ alkenyl. In some forms, the C₂-C₂₀ alkenyl can be a linear C₂-C₂₀ alkenyl, a branched C₄-C₂₀ alkenyl, a cyclic C₃-C₂₀ alkenyl, a linear or branched C₂-C₂₀ alkenyl, a linear or cyclic C₂-C₂₀ alkenyl, a branched or cyclic C₃-C₂₀ alkenyl, or a linear, branched, or cyclic C₂-C₂₀ alkenyl. Optionally, the alkenyl group can have 2-10 carbon atoms, i.e., C₂-C₁₀ alkenyl. In some forms, the C₂-C₁₀ alkenyl can be a linear C₂-C₁₀ alkenyl, a branched C₄-C₁₀ alkenyl, a cyclic C₃-C₁₀ alkenyl, a linear or branched C₂-C₁₀ alkenyl, a linear or cyclic C₂-C₁₀ alkenyl, a branched or cyclic C₃-C₁₀ alkenyl, or a linear, branched, or cyclic C₂-C₁₀ alkenyl.

The heteroalkenyl group can be linear, branched, or cyclic. It is understood that a branched heteroalkenyl or a cyclic heteroalkenyl contains at least three and two carbon atoms, respectively, in addition to at least one heteroatom. Optionally, the heteroalkenyl group can have 1-30 carbon atoms, i.e., C₁-C₃₀ heteroalkenyl. In some forms, the C₁-C₃o heteroalkenyl can be a linear C₁-C₃o heteroalkenyl, a branched C₃-C₃₀ heteroalkenyl, a cyclic C₂-C₃₀ heteroalkenyl, a linear or branched C₁-C₃₀ heteroalkenyl, a linear or cyclic C₁-C₃₀ heteroalkenyl, a branched or cyclic C₂-C₃₀ heteroalkenyl, or a linear, branched, or cyclic C₁-C₃₀ heteroalkenyl. Optionally, the heteroalkenyl group can have 1-20 carbon atoms, i.e., C₁-C₂₀ heteroalkenyl. In some forms, the C₁-C₂₀ alkenyl can be a linear C₁-C₂₀ heteroalkenyl, a branched C₃-C₂₀ heteroalkenyl, a cyclic C₂-C₂₀ heteroalkenyl, a linear or branched C₁-C₂₀ heteroalkenyl, a linear or cyclic C₁-C₂₀ heteroalkenyl, a branched or cyclic C₂-C₂₀ heteroalkenyl, or a linear, branched, or cyclic C₁-C₂₀ heteroalkenyl. Optionally, the heteroalkenyl group can have 1-10 carbon atoms, i.e., C₁-C₁₀ heteroalkenyl. In some forms, the C₁-C₁₀ heteroalkenyl can be a linear C₁-C₁₀ heteroalkenyl, a branched C₃-C₁₀ heteroalkenyl, a cyclic C₂-C₁₀ heteroalkenyl, a linear or branched C₁-C₁₀ heteroalkenyl, a linear or cyclic C₁-C₁₀ heteroalkenyl, a branched or cyclic C₂-C₁₀ heteroalkenyl, or a linear, branched, or cyclic C₁-C₁₀ heteroalkenyl.

The alkynyl group can be linear, branched, or cyclic. It is understood that a branched alkynyl contains at least four carbon atoms and that a cyclic alkynyl contains at least five carbon atoms. Optionally, the alkynyl group can have 2-30 carbon atoms, i.e., C₂-C₃₀ alkynyl. In some forms, the C₂-C₃₀ alkynyl can be a linear C₂-C₃₀ alkynyl, a branched C₄-C₃₀ alkynyl, a cyclic C₅-C₃₀ alkynyl, a linear or branched C₂-C₃₀ alkynyl, a linear or cyclic C₂-C₃₀ alkynyl, a branched or cyclic C₄-C₃₀ alkynyl, or a linear, branched, or cyclic C₂-C₃₀ alkynyl. Optionally, the alkynyl group can have 2-20 carbon atoms, i.e., C₂-C₂₀ alkynyl. In some forms, the C₂-C₂₀ alkynyl can be a linear C₂-C₂₀ alkynyl, a branched C₄-C₂₀ alkynyl, a cyclic C₅-C₂₀ alkynyl, a linear or branched C₂-C₂₀ alkynyl, a linear or cyclic C₂-C₂₀ alkynyl, a branched or cyclic C₄-C₂₀ alkynyl, or a linear, branched, or cyclic C₂-C₂₀ alkynyl. Optionally, the alkynyl group can have 2-10 carbon atoms, i.e., C₂-C₁₀ alkynyl. In some forms, the C₂-C₁₀ alkynyl can be a linear C₂-C₁₀ alkynyl, a branched C₄-C₁₀ alkynyl, a cyclic C₅-C₁₀ alkynyl, a linear or branched C₂-C₁₀ alkynyl, a linear or cyclic C₂-C₁₀ alkynyl, a branched or cyclic C₄-C₁₀ alkynyl, or a linear, branched, or cyclic C₂-C₁₀ alkynyl.

The heteroalkynyl group can be linear, branched, or cyclic. It is understood that a branched heteroalkynyl contains at least three carbon atoms and that a cyclic heteroalkynyl contains at least three carbon atoms, in addition to at least one heteroatom. Optionally, the heteroalkynyl group can have 1-30 carbon atoms, i.e., C₁-C₃₀ heteroalkynyl. In some forms, the C₁-C₃₀ heteroalkynyl can be a linear C₁-C₃₀ heteroalkynyl, a branched C₃-C₃₀ heteroalkynyl, a cyclic C₃-C₃₀ heteroalkynyl, a linear or branched C₁-C₃₀ heteroalkynyl, a linear or cyclic C₁-C₃₀ heteroalkynyl, a branched or cyclic C₃-C₃₀ heteroalkynyl, or a linear, branched, or cyclic C₁-C₃₀ heteroalkynyl. Optionally, the heteroalkynyl group can have 1-20 carbon atoms, i.e., C₁-C₂₀ heteroalkynyl. In some forms, the C₁-C₂₀ alkenyl can be a linear C₁-C₂₀ heteroalkynyl, a branched C₃-C₂₀ heteroalkynyl, a cyclic C₃-C₂₀ heteroalkynyl, a linear or branched C₁-C₂₀ heteroalkynyl, a linear or cyclic C₁-C₂₀ heteroalkynyl, a branched or cyclic C₃-C₂₀ heteroalkynyl, or a linear, branched, or cyclic C₁-C₂₀ heteroalkynyl. Optionally, the heteroalkynyl group can have 1-10 carbon atoms, i.e., C₁-C₁₀ heteroalkynyl. In some forms, the C₁-C₁₀ heteroalkynyl can be a linear C₁-C₁₀ heteroalkynyl, a branched C₃-C₁₀ heteroalkynyl, a cyclic C₃-C₁₀ heteroalkynyl, a linear or branched C₁-C₁₀ heteroalkynyl, a linear or cyclic C₁-C₁₀ heteroalkynyl, a branched or cyclic C₃-C₁₀ heteroalkynyl, or a linear, branched, or cyclic C₁-C₁₀ heteroalkynyl.

The aryl group can have 6-50 carbon atoms, i.e., C₆-C₃₀ aryl. In some forms, the C₆-C₅₀ aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl. Optionally, the aryl group can have 6-30 carbon atoms, i.e., C₆-C₃₀ aryl. In some forms, the C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Optionally, the aryl group can have 6-20 carbon atoms, i.e., C₆-C₂₀ aryl. In some forms, the C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₀ aryl, or a branched fused polycyclic C₆-C₂₀ aryl.

The heteroaryl group can have 3-50 carbon atoms, i.e., C₃-C₅₀ heteroaryl. In some forms, the C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. Optionally, the heteroaryl group can have 3-30 carbon atoms, i.e., C₃-C₃₀ heteroaryl. In some forms, the C₃-C₃₀ heteroaryl can be a branched C₃-C₃₀ heteroaryl, a monocyclic C₃-C₃₀ heteroaryl, a polycyclic C₃-C₃₀ heteroaryl, a branched polycyclic C₃-C₃₀ heteroaryl, a fused polycyclic C₃-C₃₀ heteroaryl, or a branched fused polycyclic C₃-C₃₀ heteroaryl. Optionally, the heteroaryl group can have 3-20 carbon atoms, i.e., C₆-C₂₀ heteroaryl. In some forms, the C₃-C₂₀ heteroaryl can be a branched C₃-C₂₀ heteroaryl, a monocyclic C₃-C₂₀ heteroaryl, a polycyclic C₃-C₂₀ heteroaryl, a branched polycyclic C₃-C₂₀ heteroaryl, a fused polycyclic C₃-C₂₀ heteroaryl, or a branched fused polycyclic C₃-C₂₀ heteroaryl.

Suitable therapeutic agents also include the steroids described in U.S. Pat. Nos. 5,925,630, 6,143,736, and 6,277, 838.

The therapeutic agents described herein may have one or more chiral centers and thus exist as one or more stereoisomers. Such stereoisomers can exist as a single enantiomer, a mixture of diastereomers, a racemic mixture, or combinations thereof. As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein the term “diastereomer” refers to two stereoisomers which are not mirror images but also not superimposable. The terms “racemate,” “racemic mixture” or “racemic modification” refer to a mixture of enantiomers. The term “chiral center” refers to a carbon atom to which four different groups are attached. Choice of the appropriate chiral column, eluent, and conditions necessary for effect separation of stereoisomers, such as a pair of enantiomers, is well known to one of ordinary skill in the art using standard techniques (e.g., Jacques et al., “Enantiomers, Racemates, and Resolutions”, John Wiley and Sons, Inc. 1981).

The concentration of therapeutic agent having a solubility comparable to allopregnanolone in the formulations can be between about 0.5 and about 39 mg/ml, from about 1 to about 39 mg/ml, from about 2 to about 39 mg/ml, from about 4 to about 39 mg/ml, from about 8 to about 39 mg/ml, from about 15 to about 39 mg/ml, from about 25 to about 39 mg/ml. Preferably, the concentration of the therapeutic agent in the formulations is between about 5 to about 39 mg/ml. The optimal intravenous dose in the phase 1 clinical trials was 4 mg. If the transdermal bioavailability of allopregnanolone is about 10-20% and dosing volume is 1 ml, the preferred concentration of the therapeutic agent (such as allopregnanolone) in the formulation should be between about 20 and about 35 mg/ml.

B. Carriers

The formulations include a pharmaceutically acceptable carrier. Generally, the carrier is in liquid form, in which the therapeutic agent is dissolved.

In some forms, the carrier contains water, one or more lipophilic compounds, solubilizers, a surfactant, and optionally a surfactant. In one embodiment, the carrier forms a stable microemulsion. In some forms, the therapeutic agent is dissolved in the oil phase of the microemulsion.

1. Lipophilic Compounds

In some forms, the one or more lipophilic compounds in the carrier are lipids, such as fatty acids, fatty acid esters, phospholipids, and combinations thereof. Suitable fatty acid esters include glycerides, such as monoglycerides, diglycerides, and triglycerides. The fatty acids or the fatty acid residues in the fatty acid esters can be saturated or non-saturated. The fatty acids or the fatty acid residues in the fatty acid esters can be short-chain (i.e., with an aliphatic tail having a carbon backbone of five or fewer carbon atoms), medium-chain (i.e., with an aliphatic tail having a carbon backbone of 6 to 12 carbon atoms), or long-chain (i.e., with an aliphatic tail having a carbon backbone of 13 to 21 carbon atoms). In some forms, the fatty acids or the fatty acid residues in the fatty acid esters are medium-chain, including caproic acid, caprylic acid, capric acid, and lauric acid. In some forms, the fatty acids or the fatty acid residues in the fatty acid esters are long-chain, including oleic acid and myristic acid.

In some forms, the one or more lipophilic compounds are selected from medium-chain, saturated or non-saturated, mono-, di- or tri-glycerides. In some forms, the lipophilic compounds are selected from medium-chain, saturated, mono- or di-glycerides, such as caprylic monoglyceride, caprylic diglyceride, capric monoglyceride, capric diglyceride, and combinations thereof.

In some forms, the one or more lipophilic compounds are selected from long-chain, saturated or non-saturated fatty acid or fatty acid esters, such as oleic acid and isopropyl myristate.

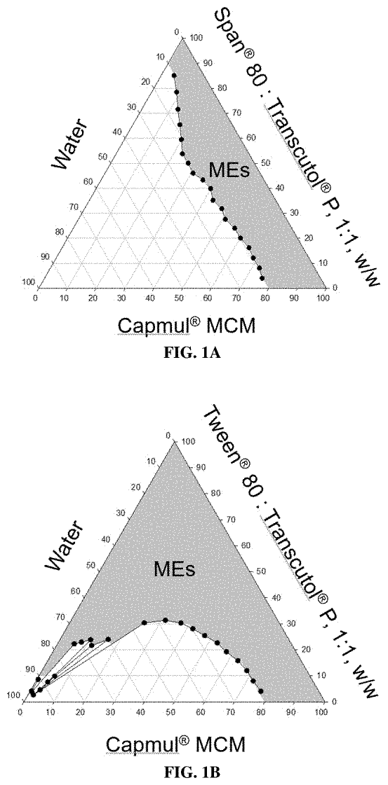

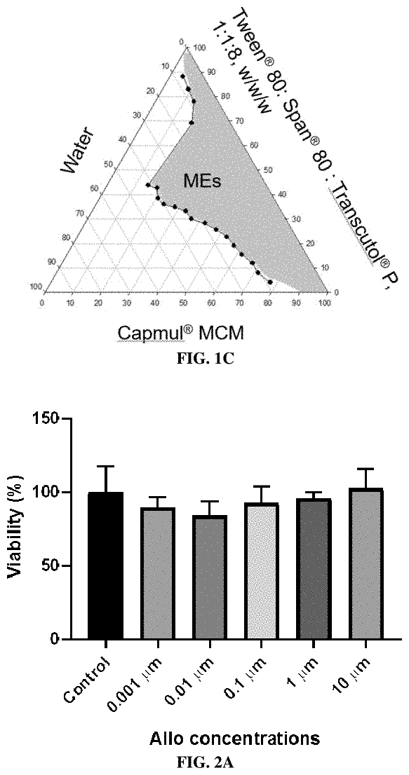

Optionally, the carrier contains an oil, wherein the lipophilic compounds originally belonged to the oil. In some forms, the oil is a natural oil such as a plant oil, e.g., coconut oil, sesame oil, olive oil, peanut oil, lavender oil, castor oil, peppermint oil, orange oil, canola oil, and corn oil. In some forms, the oil is a synthetic oil, such as CAPMUL® MCM. CAPMUL® MCM (CAS number: 91744-32-0, 26402-22-2, and 26402-26-6) is a mixture containing caprylic (ca. 70%)/capric (ca. 30%) mono- and diglycerides. In some forms, the oil is CAPMUL® MCM C_{8,} which is a mixture containing caprylic (>95%)/capric (<5%) mono- and diglycerides. In some forms, the oil is CAPMUL® MCM C_{10,} which is a mixture containing caprylic (<5%)/capric (>95%) mono- and diglycerides. Other suitable synthetic oils include CAPTEX® 300 (CAS number: 065381-09-1 and 73398-61-5; a mixture containing caprylic (ca. 70%)/capric (ca. 30%) triglycerides) and CAPMUL PG-8 (CAS number: 68332-79-6 and 31565-12-5; propylene glycol monocaprylate). The weight percent of the lipophilic compounds or the oil relative to the carrier containing SPAN®80 can be more than 7% and up to about 13%, between about 8% and about 13%, between about 9% and about 13%, between about 10% and about 13%, between about 11% and about 13%, between about 12% and about 13%, more than 7% and up to about 12%, more than 7% and up to about 11%, more than 7% and up to about 10%, more than 7% and up to about 9%, or more than 7% and up to about 8%. The ranges were determined based on microemulsion regions shown in FIG. 1A, 1B, and 1C.

The weight percent of the lipophilic compounds or the oil relative to the carrier containing TWEEN®80 can be more than 0.01% and up to about 13%.

The weight percent of the lipophilic compounds or the oil relative to the carrier containing both TWEEN®80 and SPAN®80 can be more than 0.05% and up to about 13%, or example, between about 8% and about 13%, or more than 7% and up to about 10%. The weight percent is the preferred weight percent ranges that are selected from the microemulsion regions shown in FIG. 1. Preferably, the weight percent of the lipophilic compounds or the oil relative to the carrier is between about 7% and about 15%, between about 10% and about 13%, or between about 7% and about 11%, .

2. Surfactant and Co-surfactant

The surfactant can be anionic, cationic, nonionic, or zwitterionic. In certain embodiments, the surfactant is a non-ionic surfactant, such as but not limited to, TWEEN® surfactants (polysorbates), such as TWEEN® 20 (Polysorbate 20), TWEEN® 65 (Polysorbate 65), and TWEEN® 80 (Polysorbate 80); SPAN® surfactants (sorbitan alkanoates), such as SPAN® 20 (sorbitan monolaurate), SPAN® 60 (sorbitan monostearate), SPAN® 65 (sorbitan tristearate), SPAN® 80 (sorbitan monooleate); polyoxyethylene fatty acid esters, such as CREMOPHOR® EL (PEG-35 castor oil) and CREMOPHOR® RH 40 (PEG-40 castor oil); and combinations thereof.

In some forms, the surfactant is sorbitan monooleate, Polysorbate 80 or a combination of sorbitan monooleate and Polysorbate 80. The weight ratio of sorbitan monooleate to Polysorbate 80 in the combination can be between 0.5 and about 2, such as about 1.

The carrier can also contain a co-surfactant, which can increase the lipid-solubilizing capacity of the microemulsion. Exemplary co-surfactants include short-chain (e.g., C₂-C₅), medium-chain (e.g., C₆-C₁₂), and long-chain (e.g., C₁₃-C₂₁) alcohols or amines, wherein one or more carbon atoms on the backbone of the carbon chain can be substituted by a heteroatom, independently selected from oxygen, nitrogen, or sulfur. In some forms, the co-surfactants is diethylene glycol monoethyl ether.

The weight ratio between the surfactant and the co-surfactant in the carrier can between 1:20 to 20:1, from 1:10 to 10:1, from 1:5 to 5:1, from 1:2 to 2:1, about 1:1, about 1:3, or about 1:4.

As shown by FIGS. 1A, 1B, and 1C, the weight percent of each component that forms microemulsions is very broad. The weight percent of the surfactant containing SPAN®80 or the combination of the surfactant containing SPAN®80 and the co-surfactant relative to the carrier can be between about 50% and about 90%, preferably between about 74% and about 88%.

Preferably, the weight percent of the surfactant containing SPAN®80 or the combination of the surfactant containing SPAN®80 and the co-surfactant relative to the carrier is between about 85% and about 88% when the weight percent of the lipophilic compounds or the oils is between about 7% and about 8%, or between about 73% and about 84% when the weight percent of the lipophilic compounds or the oils is between about 12% and about 13%.

The weight percent of the surfactant containing TWEEN®80 or the combination of the surfactant containing TWEEN®80 and the co-surfactant relative to the carrier can be between about 12% and about 30%, between about 18% and about 30%, or between about 21% and about 30%.

Preferably, the weight percent of the surfactant containing TWEEN®80 or the combination of the surfactant containing TWEEN®80 and the co-surfactant relative to the carrier is between about 12% and about 30% when the weight percent of the lipophilic compounds or the oils is between about 0.01% and about 1.6%, between about 13% and about 30% when the weight percent of the lipophilic compounds or the oils is between about 1.6% and about 2%, between about 16% and about 30% when the weight percent of the lipophilic compounds or the oils is between about 2% and about 3%, or between about 27% and about 30% when the weight percent of the lipophilic compounds or the oils is between about 3% and about 13%.

The weight percent of the surfactant containing both TWEEN®80 and SPAN®80 or the combination of the surfactant containing both TWEEN®80 and SPAN®80 and the co-surfactant relative to the carrier can be between about 81% and about 87%, or between about 85% and about 86%,

Preferably, the weight percent of the surfactant containing TWEEN®80 and SPAN®80 or the combination of the surfactant containing TWEEN®80 and SPAN®80 and the co-surfactant relative to the carrier is about 81% when the weight percent of the lipophilic compounds or the oils is between about 12% and about 13%, about 82% when the weight percent of the lipophilic compounds or the oils is between about 11% and about 13%, about 83% when the weight percent of the lipophilic compounds or the oils is between about 10% and about 13%, about 85% when the weight percent of the lipophilic compounds or the oils is between about 8% and about 13%, or about 87% when the weight percent of the lipophilic compounds or the oils is between about 7% and about 12%.

3. Other Components of the Carrier

The carrier generally contains water, optionally in the form of an aqueous solution. The aqueous solution may contain one or more buffering agents, such as TRIS, phosphate, borate, HEPES, MOPS, and MES. In some forms, the aqueous solution has a pH in the range from about 5 to about 9, from about 5.5 to about 8.5, or from about 6 to about 8. For example, the buffered aqueous solution can be phosphate-buffered saline (pH 6.8-7.6). The aqueous solution may contain one or more tonicity-adjusting agents such as salts (e.g., sodium chloride, potassium chloride, sodium lactate, calcium chloride, sodium sulfate) and hydrophilic compounds (e.g., glycerol, glucose, lactose, mannitol, propylene glycol). The weight percent of water or the aqueous solution relative to the carrier containing SPAN®80 can be more than 4% and up to about 14%, between about 7% and about 14%, between about 8% and about 12%, between about 9% and about 13%, between about 10% and about 14%, between about 11% and about 14%, between about 12% and about 14%, between about 13% and about 14%, more than 4% and up to about 13%, more than 4% and up to about 9%, more than 4% and up to about 7%, or more than 4% and up to about 6%.

Preferably, the weight percent of water or the aqueous solution relative to the carrier containing SPAN®80 is more than 5% and up to about 8% when the weight percent of the lipophilic compounds or the oils is between about 7% and about 8%, between about 4% and about 10% when the weight percent of the lipophilic compounds or the oils is between about 8% and about 10%, between about 4% and about 12% when the weight percent of the lipophilic compounds or the oils is between about 11% and about 13%.

The weight percent of water or the aqueous solution relative to the carrier containing TWEEN®80 can be more than 57% and up to about 88%, between about 72% and about 88%, or between about 78% and about 88%.

Preferably, the weight percent of water or the aqueous solution relative to the carrier containing TWEEN®80 is more than 57% and up to about 70% when the weight percent of the lipophilic compounds or the oils is between about 3% and about 13 or between about 68% and about 88% when the weight percent of the lipophilic compounds or the oils is between about 0.01% and about 1.5%.

The weight percent of water or the aqueous solution relative to the carrier containing TWEEN®80 and SPAN®80 can be more than 1% and up to about 7%, or between about 4% and about 7.

Preferably, the weight percent of water or the aqueous solution relative to the carrier containing TWEEN®80 and SPAN®80 is more than 1% and up to about 6% when the weight percent of the lipophilic compounds or the oils is between about 7% and about 12%, between about 5% and about 7% when the weight percent of the lipophilic compounds or the oils is between about 11% and about 13%, or between about 6% and about 7% when the weight percent of the lipophilic compounds or the oils is between about 12% and about 13%.

In some forms, the carrier also contains a transdermal penetration enhancer. Exemplary transdermal penetration enhancers include sulphoxides (such as dimethylsulphoxide), azones (such as laurocapram), pyrrolidones (such as 2-pyrrolidone), alcohols and alkanols (such as ethanol and decanol), glycols (such as propylene glycol), polyols (such as glycerol), surfactants, and terpenes. In some forms, the transdermal penetration enhancer is ethanol, propylene glycol, or glycerol. In some forms, the transdermal penetration enhancer is ethanol.

The weight percent of the transdermal penetration enhancer relative to the carrier can be up to about 20%, between about 10% and about 20%, between about 12% and about 20%, or between about 14% and about 20%.

4. Properties of the Carrier

The carrier forms a stable microemulsion. The structure of the microemulsion system, o/w, w/o, or bicontinuous, can be predicted by the hydrophilic-lipophilic balance (HLB) of emulsifiers. In general, low HLB (3-6) surfactants tend to form w/o microemulsion system whereas high HLB (8-18) surfactants are preferred to form o/w microemulsions (Lawrence and Rees, Adv Drug Deliv Rev, 2000, 45:89-121). The surfactant and co-surfactant of the first microemulsions in FIG. 1B are polysorbate 80 (Tween® 80, HLB=15) and diethylene glycol monoethyl ether (Transcutol® P, HLB=-4) and mixed 1:1 by weight percent. Based on the HLB values of the surfactant and co-surfactant and their weight percent, the estimated HLB of the first microemulsions is 9.5, and thus the predicted structure of the first microemulsions is o/w microemulsion system. The second microemulsion system presented in FIG. 1A contains sorbitan monooleate (Span® 80, HLB=4.3), which is mixed with diethylene glycol monoethyl ether by 1:1 (weight percent). The predicted structure of the second microemulsions is w/o microemulsion system as the estimated HLB of the second microemulsions is 4.15. The surfactants and co-surfactant of the third microemulsion system in FIG. 1C are composed of polysorbate 80, sorbitan monooleate and diethylene glycol monoethyl ether at 1:1:8 (by weight percent), and the predicted structure of ME-C is w/o system as the estimated HLB of ME-C is 5.13.The microemulsion formed of the carrier can be direct (the oil phase dispersed in the aqueous phase, o/w), reversed (the aqueous phase dispersed in the oil phase, w/o), or bicontinuous microemulsion.

In some forms, the microemulsion has a viscosity of between about 1 and about 20 centipoise (cP), between about 2 and about 15 cP, between about 5 and about 15 cP, or between about 5 and about 10 cP.

In some forms, the microemulsion is stable at room temperature (around 20-25° C.) and 50% relative humidity for at least a month without precipitation of the therapeutic agent, color change of the formulation, or transparency change of the formulation. In some forms, the microemulsion is stable at 40° C. and 75% relative humidity for at least a month without precipitation of the therapeutic agent, color change of the formulation, or transparency change of the formulation.

In some forms, the solubility of the therapeutic agent in the carrier at room temperature is at least about 6-fold, at least about 10-fold, at least at least about 15-fold, or at least about 26-fold higher than the solubility of the allopregnanolone solution for the intravenous and intramuscular administration (1.5 mg/ml in 6% cyclodextrin solution) in phase 1 clinical trials a corresponding carrier without the one or more lipophilic compounds, surfactant, or co-surfactant at room temperature.

In some forms, the permeability of the therapeutic agent from the carrier is characterized by a flux coefficient of at least 15 μg/cm²/h, at least 25 μg/cm²/h, at least 45 μg/cm²/h, or at least 55 μg/cm²/h, or at least 60 μg/cm²/h. For example, the flux coefficient can be between about 10 and about 60 μg/cm²/h.

Permeability coefficient to evaluate the effect of potential penetration enhancers (ethanol and propylene glycol). In some forms, the permeability coefficient of the therapeutic agent from the carrier, such as ethanol and propylene glycol, is characterized by a permeability coefficient of at least 3.00×10⁻³ cm/h, at least 5.0×10⁻³ cm/h, or at least 6.00×10⁻³ cm/h. For example, the permeability coefficient can be between about3.50×10⁻³ and about 6.00×10⁻³ cm/h.

The permeability of the therapeutic agent can be measured against a 300 μm-thick START-M® membrane at 32° C. The receiver medium can be phosphate buffered saline, optionally supplemented with 10% (w/v) 2-hydroxypropyl-β-cyclodextrin (HβCD).

5. Solubilizers

Separate from the surfactants and/or co-surfactants described above, the carrier can also contain solubilizers, preferably solubilizers of 3a-hydroxy-5a-pregnan-20-one (allopregnanolone), a derivative or analogue thereof, or a pharmaceutically acceptable salt of the derivative or analogue. Preferably, solubilizers increase the solubility of the 3a-hydroxy-5a-pregnan-20-one (allopregnanolone), a derivative or analogue thereof, or a pharmaceutically acceptable salt of the derivative or analogue in the carrier, compared to a corresponding carrier that does not contain the solubilizer. Preferably, 3a-hydroxy-5a-pregnan-20-one, a derivative or analogue thereof, or a pharmaceutically acceptable salt of the derivative or analogue is dissolved in the solubilizers within the carriers. Preferably, a predominant amount of the 3-hydroxy-5a-pregnan-20-one, a derivative or analogue thereof, or a pharmaceutically acceptable salt of the derivative or analogue is dissolved in the solubilizers within the carrier, than in other components of the carrier. “Predominant” refers to more than 50% of the 3-hydroxy-5a-pregnan-20-one, a derivative or analogue thereof, or a pharmaceutically acceptable salt of the derivative or analogue in the carrier. For example, more than 50% of the 3-hydroxy-5a-pregnan-20-one, a derivative or analogue thereof, or a pharmaceutically acceptable salt of the derivative or analogue can be dissolved in the solubilizers, compared to the surfactant, co-surfactant, or both. In some forms, the solubilizer solubilizes the a derivative or analogue thereof, or a pharmaceutically acceptable salt of the derivative or analogue via encapsulation. In some forms, the solubilizer solubilizes the 3-hydroxy-5a-pregnan-20-one, a derivative or analogue thereof, or a pharmaceutically acceptable salt of the derivative or analogue by forming a host-guest complex.

The solubilizers can be selected from: polysaccharides; water-soluble organic solvents; non-ionic surfactants that can enhance hydroxy-5a-pregnan-20-one solubility; water-insoluble lipids; organic liquids/semi-solids; phospholipids; and combinations thereof. A general review of solubilizing excipients in oral and injectable formulations is provided by Strickley, Pharmaceutical Research, 2004, 21(2), 201-230, the contents of which are hereby incorporated in their entirety, by reference.

Polysaccharides include, but are not limited to, cyclodextrins, derivatives of cyclodextrins, chemically modified cellulose (such as hydroxypropyl methyl cellulose), and combinations thereof. The cyclodextrins and/or derivatives thereof, can be selected from: α-cyclodextrins, β-cyclodextrins, γ-cyclodextrins, hydroxypropyl-β-cyclodextrins (such as 2-hydroxypropyl-β-cyclodextrin), sulfobutylether-β-cyclodextrins, heptakis-6-sulfoethylsulfanyl-6-deoxy-β-cyclodextrin, heptakis-6-methylsulfanyl-6-deoxy-2-(2-(2-(2-methoxyethoxy)ethoxy)ethyl)-β-cyclodextrins, heptakis-6-thioglyceryl-6-deoxy-β-cyclodextrins, and combinations thereof. In some forms, the cyclodextrins can be β-cyclodextrins. In some forms, the β-cyclodextrins can be hydroxypropyl-β-cyclodextrins (such as 2-hydroxypropyl-β-cyclodextrin).

Water-soluble organic solvents include, but are not limited to, polyethylene glycol 300, polyethylene glycol 400, ethanol, propylene glycol, glycerin, N-methyl-2-pyrrolidone, dimethylacetamide, dimethylsulfoxide, and combinations thereof.

Non-ionic surfactants that can enhance hydroxy-5a-pregnan-20-one solubility include, but are not limited to, Cremophor EL, Cremophor RH 40, Cremophor RH 60, d-α-tocopherol polyethylene glycol 1000 succinate, polysorbates (such as polysorbate 20, polysorbate 80, etc), poly(ethylene glycol) 12-hydroxystearate, sorbitan monooleate, poloxamer (e.g. poloxamer 407), Labrafil M-1944CS, Labrafil M-2125CS, Labrasol, Gellucire 44/14, capric glycerides, mono- and di-fatty acid esters of PEG 300, 400, or 1750, and combinations thereof.

Water-insoluble lipids include, but are not limited to, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, and combinations thereof.

Organic liquids/semi-solids include, but are not limited to, beeswax, d-α-tocopherol, oleic acid, medium-chain mono- and diglycerides, and combinations thereof.

Phospholipids include, but are not limited to, hydrogenated soy phosphatidylcholine, distearoylphosphatidylglycerol, L-α-dimyristoylphosphatidylcholine, L-α-dimyristoylphosphatidylglycerol, and combinations thereof.

In some forms, the carrier is as described above, except that the weight percent of the solubilizers relative to the carrier is between about 10% and about 90%, between about 60% and about 90%, between about 73% and 88%, or between about 80% and about 85%.

C. Other Therapeutic, Prophylactic or Diagnostic Agents

In addition to the therapeutic agent described above, the formulations can further contain one or more therapeutic, prophylactic or diagnostic agent(s). The additional agent can be dissolved or dispersed in the carrier. In some forms, it is dissolved in the aqueous phase of the microemulsion formed by the carrier. In some forms, it is dissolved in the oil phase of the microemulsion formed by the carrier.

In certain embodiments, the additional agent is a steroid. Suitable steroids include biologically active forms of vitamin D3 and D2, such as those described in U.S. Pat. Nos. 4,897,388 and 5,939,407. Such steroids may be co-administered with the therapeutic agent to further aid in neurogenic stimulation or induction and/or prevention of neural loss, particularly for treatments of Alzheimer's disease. Suitable steroids also include biologically active forms of estrogen and estrogen. Such steroids may be co-administered with the therapeutic agent to enhance neuroprotection as described in Brinton (2001) Learning and Memory 8 (3): 121-133. Other neuroactive steroids, such as various forms of dehydroepiandrosterone (DHEA) as described in U.S. Pat. No. 6,552,010, can also be co-administered with the therapeutic agent to further aid in neurogenic stimulation or induction and/or prevention of neural loss.

Agents that cause neural growth and outgrowth of neural networks, such as nerve growth factor (NGF) and brain-derived neurotrophic factor (BDNF), can be administered either simultaneously with or before or after the administration of the therapeutic agent. Additionally, inhibitors of neural apoptosis, such as inhibitors of calpains and caspases and other cell death mechanisms, such as necrosis, can be co-administered with the therapeutic agent to further prevent neural loss associated with certain neurological diseases and neurological defects.

D. Formulations

The formulations generally contain a therapeutic and/or prophylactic agent and a pharmaceutically acceptable carrier, wherein the therapeutic agent is dissolved in the pharmaceutically acceptable carrier. In some forms, the carrier of the formulations contains water, one or more lipophilic compounds as described above, a surfactant as described above, optionally a co-surfactant as described above, and optionally a tissue penetration enhancer as described above. In certain embodiments, the carrier of the formulations contains water, one or more lipophilic compounds as described above, a surfactant as described above, a co-surfactant as described above, and a tissue penetration enhancer as described above. The concentration ranges of the therapeutic agent may depend on dose regimen to achieve a therapeutic concentration level after administration and the maximum solubility of the therapeutic agent in the formulations. The weight percent ranges of inactive ingredients (oil/lipophilic compounds, surfactant(s), co-surfactant, and a penetration enhancer) may depend on multiple factors including the stability of microemulsions, in vitro/in vivo permeability of the therapeutic agent, and safety levels for clinical uses.

As may be understood by those skilled in the art, the dosage of the therapeutic agent in the formulations can be effective to stimulate or induce neural regeneration or neurogenesis, protect against neural loss, or ameliorate one or more symptoms associated with Alzheimer's disease or other neurodegenerative diseases in a subject in need thereof. For example, the dosage of the therapeutic agent, e.g., 3α-hydroxy-5α-pregnan-20-one, a derivative or analogue thereof, or a pharmaceutically acceptable salt of the derivative or analogue, can be in the range of about 4 to about 50 mg, about 15 to about 35 mg, about 20 to about 30 mg, or about 25mg. The formulation may contain a single dose or a plurality of doses of the therapeutic agent.

1. Exemplary Formulations

i. Exemplary Formulations with Tissue Penetration Enhancer

Exemplary formulations contain a therapeutic agent and a carrier containing water, one or more lipophilic compounds, a surfactant, a co-surfactant, and a tissue penetration enhancer.

In some embodiments, the therapeutic agent is 3a-hydroxy-5a-pregnan-20-one; the one or more lipophilic compounds are caprylic monoglyceride, caprylic diglyceride, capric monoglyceride, capric diglyceride, or combinations thereof; the surfactant is sorbitan monooleate, Polysorbate 80, or a combination of sorbitan monooleate and Polysorbate 80 at a weight ratio of about 1; the co-surfactant is diethylene glycol monoethyl ether; and the transdermal penetration enhancer is ethanol. Optionally, the carrier contains CAPMUL® MCM, wherein the one or more lipophilic compounds originally belonged to the CAPMUL® MCM.

In some embodiments, the carrier of these formulations contains the following:

(a) CAPMUL® MCM at a weight percent of more than 0.01% and up to 130, preferably between about 7% and about 13%, relative to the carrier;

(b) a surfactant and a co-surfactant at a total weight percent of between about 12% and about 88%, preferably between about 73% and about 88%, between about 12% and about 30%, or between about 81% and about 87%, relative to the carrier, wherein the surfactant is sorbitan monooleate, Polysorbate 80, or a combination of sorbitan monooleate and Polysorbate 80 at a weight ratio of about 1, and wherein the co-surfactant is diethylene glycol monoethyl ether;

(c) a transdermal penetration enhancer at a weight percent of more than 0% and up to about 20% relative to the carrier, wherein the transdermal penetration enhancer is ethanol; and

(d) water at a weight percent of more than 1% and up to about 88%, preferably between about 4% and about 14%, between about 57% and about 88%, or between about 1% and about 7%, relative to the carrier.

One exemplary carrier contains the following:

(a) CAPMUL® MCM at a weight percent of about 8% relative to the carrier;

(b) a surfactant and a co-surfactant both at a weight percent of about 34% relative to the carrier, wherein the surfactant is sorbitan monooleate and the co-surfactant is diethylene glycol monoethyl ether;

(c) a transdermal penetration enhancer at a weight percent of about 20% relative to the carrier, wherein the transdermal penetration enhancer is ethanol; and

(d) water at a weight percent of about 4% relative to the carrier.

Another exemplary carrier contains the following:

(a) CAPMUL® MCM at a weight percent of about 13% relative to the carrier;

(b) a surfactant and a co-surfactant at a weight percent of about 16% and about 50% relative to the carrier, respectively, wherein the surfactant is a combination of sorbitan monooleate and Polysorbate 80 at a weight ratio of about 1 and the co-surfactant is diethylene glycol monoethyl ether;

(c) a transdermal penetration enhancer at a weight percent of about 15% relative to the carrier, wherein the transdermal penetration enhancer is ethanol; and

(d) water at a weight percent of about 6% relative to the carrier.

Another exemplary carrier contains the following:

(a) CAPMUL® MCM at a weight percent of about 13% relative to the carrier;

(b) a surfactant and a co-surfactant both at a weight percent of about 15% relative to the carrier, wherein the surfactant is Polysorbate 80 and the co-surfactant is diethylene glycol monoethyl ether;

(c) a transdermal penetration enhancer at a weight percent of about 15% relative to the carrier, wherein the transdermal penetration enhancer is ethanol; and

(d) water at a weight percent of about 42% relative to the carrier.

ii. Exemplary Formulations Without Tissue Penetration Enhancer

Exemplary formulations can contain a therapeutic agent and a carrier containing water, one or more lipophilic compounds, a surfactant, and a co-surfactant.

In some embodiments, the therapeutic agent is 3a-hydroxy-5a-pregnan-20-one; the one or more lipophilic compounds are caprylic monoglyceride, caprylic diglyceride, capric monoglyceride, capric diglyceride, or a combination thereof; the surfactant is sorbitan monooleate or Polysorbate 80; and the co-surfactant is diethylene glycol monoethyl ether. Optionally, the carrier contains CAPMUL® MCM, wherein the one or more lipophilic compounds originally belonged to the CAPMUL® MCM.

In some embodiments, the carrier contains the following: (a) CAPMUL® MCM at a weight percent of more than 0.01% and up to 13%, preferably between about 7% and about 13%, relative to the carrier;

(b) a surfactant and a co-surfactant at a total weight percent of between about 12% and about 88%, preferably between about 73% and about 88%, between about 12% and about 30%, or between about 81% and about 87%, relative to the carrier, wherein the surfactant is sorbitan monooleate or Polysorbate 80, and wherein the co-surfactant is diethylene glycol monoethyl ether; and

(c) water at a weight percent of more than 1% and up to about 88%, preferably between about 4% and about 14%, between about 57% and about 88%, or between about 1% and about 7%, relative to the carrier.

One exemplary carrier contains the following:

(a) CAPMUL® MCM at a weight percent of about 2% relative to the carrier;

(b) a surfactant and a co-surfactant at a total weight percent of about 30% relative to the carrier, wherein the surfactant is Polysorbate 80 and the co-surfactant is diethylene glycol monoethyl ether, and wherein the surfactant and the co-surfactant are at a weight ratio of about 1:1; and

(c) water at a weight percent of about 68% relative to the carrier.

Another exemplary carrier contains the following:

(a) CAPMUL® MCM at a weight percent of about 13% relative to the carrier;

(b) a surfactant and a co-surfactant at a total weight percent of about 81% relative to the carrier, wherein the surfactants are Polysorbate 80 and sorbitan monooleate, and the co-surfactant is diethylene glycol monoethyl ether, and wherein the surfactants and the co-surfactant are at a weight ratio of about 1:1:8; and

(c) water at a weight percent of about 6% relative to the carrier.

Another exemplary carrier contains the following:

(a) CAPMUL® MCM at a weight percent of about 13% relative to the carrier;

(b) a surfactant and a co-surfactant at a total weight percent of about 73% relative to the carrier, wherein the surfactant is sorbitan monooleate and the co-surfactant is diethylene glycol monoethyl ether, and wherein the surfactant and the co-surfactant are at a weight ratio of about 1:1; and

(c) water at a weight percent of about 14% relative to the carrier.

2. Self-Emulsifying Compositions

In some embodiments, the formulations are generated using self-emulsifying compositions. The self-emulsifying compositions contain all the components of the formulations except water. Upon exposure to an aqueous environment, contact between the aqueous medium and the self-emulsifying compositions generates microemulsion, thereby creating the formulations. Preferably, no mixing force is required to generate the microemulsion.

The self-emulsifying compositions can be encapsulated in capsules (soft shell or hard shell). When the capsule is exposed to an aqueous environment and the capsule shell dissolves, contact between the aqueous medium and the self-emulsifying composition within the capsule generates microemulsion, thereby creating the corresponding formulation.

The self-emulsifying compositions are useful for sublingual delivery.

### III. Methods of Making

The formulations can be readily prepared using techniques generally known to those skilled in the art.

In certain embodiments, the carrier is prepared by mixing the components of the carrier, optionally under stirring. For example, the carrier can be generated by adding water, optionally in a plurality of increments, to the rest of the components of the carrier, under stirring.

In certain embodiments, the therapeutic agent is incorporated into the formulation by mixing it with the carrier, optionally under stirring. In certain embodiments, the therapeutic agent is mixed with the lipophilic compounds first, the mixture of which is then combined with other components to generate the formulations.

In certain embodiments, the formulations are generated by mixing the corresponding self-emulsifying compositions with an aqueous medium such as water. Optionally, the formulations can be generated in situ by directly administering the corresponding self-emulsifying compositions to a subject in need thereof.

In the preferred embodiment, the therapeutic agent is first dissolved in the lipophilic phase. The surfactant(s) and co-surfactants are incorporated into the lipophilic component. An aqueous medium such as water is added to the mixture of the lipophilic component, surfactant(s), and co-surfactant, which forms clear and isotropic microemulsions. Lastly, a penetration enhancer such as ethanol is optionally incorporated into the microemulsions. Between each step, the mixture is optionally stirred, vortexed or gently shaken.

Microemulsions generally do not require high energy input, such as homogenizers or ultrasound generators, since it spontaneously forms clear and isotropic microemulsions even after gentle shaking.

The microneedles and substrate can be made by methods known to those skilled in the art. Examples include microfabrication processes, by creating small mechanical structures in silicon, metal, polymer, and other materials. Three-dimensional arrays of hollow microneedles can be fabricated, for example, using combinations of dry etching processes; micromold creation in lithographically-defined polymers and selective sidewall electroplating; or direct micromolding techniques using epoxy mold transfers. These methods are described, for example, in U.S. Pat. Nos. 6,334,856, 6,503,231, 6,611,707, 8,708,966, 10,265,511; in PCT patent application publication WO 2011/076537; Henry, et al., Micro Electro Mechanical Systems, Heidelberg, Germany, 1998, 494-98; Li et al., Curr Med Chem, 2017, 24(22):2413-2422; Cheung et al., Drug Delivery, 2016, 7, 2338-2354; and references cited therein.

A. Kits and Devices

The compositions can be packaged in a kit. The kit can be a dosage unit kit containing a single dose or a plurality of doses of a formulation disclosed herein. The kit may include instructions for use.

In certain embodiments, the formulation may be placed in a sealed container such as a glass or plastic vial or bottle, encompassed in a delivery vehicle or device, or encapsulated in a capsule (soft shell or hard shell).

In certain embodiments, the kit may contain one or more containers for dry components and one or more containers for liquid components, which are mixed together to form a formulation disclosed herein before administration to a subject in need thereof.

In certain embodiments, the kit may contain a self-emulsifying composition as described above. The self-emulsifying composition may be encapsulated in a capsule (soft shell or hard shell).

The kits are generally designed and adapted for topical use, transdermally or transcutaneously. They may contain one or more delivery vehicles or devices specific for the approach of administration, such as microneedles for microneedle administration, spray bottle or syringe for intranasal or sublingual administration, film for buccal administration, and capsule for sublingual administration. The formulations or self-emulsifying compositions may be placed in the delivery vehicles or devices from the manufacturer or added to the delivery vehicles or devices before administration to a subject in need thereof.

An exemplary kit includes a formulation disclosed herein, which contains one or more dosages of between about 2 and about 10 mg of the therapeutic agent, such as 3a-hydroxy-5a-pregnan-20-one, a derivative or analogue thereof, or a pharmaceutically acceptable salt of the derivative or analogue. The kit may also include instructions for administering a single dose of the therapeutic agent once per week or less frequently. The instructions can be affixed to the packaging material or can be included as a package insert. While the instructions typically contain written or printed materials, they are not limited to such. As used herein, the term “instructions” can include the address of an internet site that provides the instructions.

### IV. Microneedle Devices

Alternatively, the formulation can be administered using a microneedle device, such as a microneedle patch, to a subject in need thereof. The microneedle device generally includes at least two components: a plurality of microneedles and a substrate to which the base of the microneedles is secured or integrated, and typically a reservoir for drug.

The microneedles can be dissolvable or biodegradable. In some forms, the microneedles dissolve upon contact with a biofluid, such as interstitial fluid, intravascular fluid, and cerebrospinal fluid). In some forms, the microneedles biodegrade after penetration through the skin.

By selecting the materials and/or adjusting the physical properties of the microneedles, the microneedle device can be designed as an immediate release device, a controlled release device, or both. In some forms, the microneedle device provides an immediate release of a single dose of the formulation. In some forms, the microneedle device provides a controlled release of one or more doses of the formulation over a certain period, such as about 5 min, 10 min, 20 min, 30 min, 40 min, 50 min, one hour, two hours, three hours, four hours, five hours, six hours, eight hours, ten hours, and up to days. In some forms, the microneedle device provides an immediate release of the formulation, followed by sustained release of the formulation for a certain period as exemplified above.

In some forms, the formulation is encapsulated in the microneedles, which serve as individual reservoirs. In some forms, the microneedle device further contains at least one reservoir that is not a microneedle, which is in connection (selectably in fluid connection) preferably with the base end of one or more of the microneedles, either integrally or separably until the moment of use.

In some forms, the microneedles are provided as a multi-dimensional array, in contrast to a microneedle device with a single microneedle or single row of microneedles. The microneedle devices can be adapted to be a single-use, disposable device, or can be adapted to be fully or partially reusable.

Exemplary microneedle devices can be found in U.S. Pat. Nos. 6,334,856, 6,503,231, 6,611,707, 8,708,966, 10,265,511; in PCT patent application publication WO 2011/076537; Henry, et al., Micro Electro Mechanical Systems, Heidelberg, Germany, 1998, 494-98; Li et al., Curr Med Chem, 2017, 24(22):2413-2422; Cheung et al., Drug Delivery, 2016, 7, 2338-2354; and references cited therein.

A. Microneedles

The microneedles can be hollow. In some forms, each microneedle contains at least one substantially annular bore or channel, optionally having a diameter large enough to permit passage of the formulation through the microneedle. The hollow shafts may be linear, i.e., extend upwardly from needle base to needle tip, or they may take a more complex path, e.g., extend upwardly from the needle base, but then lead to one or more ‘portholes’ or ‘slits’ on the sides of the needles, rather than an opening at the needle tip. In some forms, the microneedles can be sterilizable using standard methods such as ethylene oxide or gamma irradiation.

The microneedles can be constructed from a variety of materials, including metals, ceramics, semiconductors, organics, polymers, and composites. Preferred materials of construction include pharmaceutical grade stainless steel, gold, titanium, nickel, iron, tin, chromium, copper, palladium, platinum, alloys of these or other metals, silicon, silicon dioxide, polymers, and combinations thereof. Representative biodegradable polymers include polymers of hydroxy acids such as lactic acid and glycolic acid, polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polyester, and polyacrylamides. In some forms, the microneedles are made of one or more materials that are dissolvable upon contact with a biofluid. Such materials include polysaccharides and derivatives thereof (e.g., hyaluronate, chitosan, dextran, chondroitin sulfate, carboxymethyl cellulose (CMC), maltodextrin), oligosaccharides or monosaccharides (e.g., sucrose, trehalose, lactose, sorbitol), hydrophilic or amphiphilic polymers (e.g., polyvinylpyrrolidone (PVP), poly(vinyl alcohol) (PVA), polyacrylic acid (PAA), GANTREZ™ AN polymers AN-119, AN-139, AN-149, and AN-169 (maleic anhydride polymers and copolymers), gelatin), and small molecules (e.g., threonine or other amino acids).

In some forms, the microneedles have the mechanical strength to remain intact while being inserted into the biological barrier (e.g., skin), while remaining in place for a certain period, such as about 5 min, 10 min, 20 min, 30 min, 40 min, 50 min, one hour, two hours, three hours, four hours, five hours, six hours, eight hours, ten hours, and up to days, and while being removed. In some forms where the microneedles are formed of one or more biodegradable polymers, the microneedles must remain intact at least long enough for the microneedle to serve its intended purpose (e.g., its conduit function for delivery of the formulation).

In some forms, the microneedles, especially the tips of the microneedles, can be cracked or shattered during and/or after being inserted into the biological barrier, thereby immediately releasing the encapsulated formulation into the target site. The cracked microneedles or pieces of the shattered microneedles can be dissolved upon contact with a biofluid or biodegraded in situ.

The microneedles can have straight or tapered shafts. In a preferred form, the diameter of the microneedle is greatest at the base end of the microneedle and tapers to a point at the end distal to the base. The microneedle can also be fabricated to have a shaft that includes both a straight (untapered) portion and a tapered portion. The needles may also not have a tapered end at all, i.e., they may simply be cylinders with blunt or flat tips. A hollow microneedle that has a substantially uniform diameter, but which does not taper to a point, is referred to herein as a “microtube.” As used herein, the term “microneedle” includes both microtubes and tapered needles unless otherwise indicated.

The microneedles can be oriented perpendicular or at an angle to the substrate. Preferably, the microneedles are oriented perpendicular to the substrate so that a larger density of microneedles per unit area of substrate can be provided.

The microneedles can be formed with shafts that have a circular cross-section in the perpendicular, or the cross-section can be non-circular. For example, the cross-section of the microneedle can be polygonal (e.g., star-shaped, square, triangular), oblong, or another shape. The shaft can have one or more bores. The cross-sectional dimensions typically are between about 1 μm and 500 μm, and preferably between 10 and 100 μm. The outer diameter is typically between about 10 μm and about 100 μm, and the inner diameter is typically between about 3 μm and about 80 μm.

In some forms, the cross-sectional dimensions are designed to leave a residual hole (following microneedle insertion and withdrawal) of less than about 0.2 μm, to avoid making a hole which would allow bacteria to enter the penetration wound. The actual microneedle diameter will typically be in the few micron range, since the holes typically contract following withdrawal of the microneedle. Larger diameter and longer microneedles are acceptable, so long as the microneedle can penetrate the biological barrier to the desired depth.

The length of the microneedles typically is between about 10 μm and 1 mm, preferably between 100 μm and 800 μm, between 100 μm and 500 μm, and more preferably between 150 μm and 350 μm or between 450 μm and 650 μm. The length is selected for the particular application, accounting for both an inserted and uninserted portion. In transdermal or transcutaneous applications, the “insertion depth” of the microneedles is preferably less than about 100-150 μm or less than about 550-650 μm, so that insertion of the microneedles into the skin does not penetrate into the dermis or does not deeply penetrate into the dermis, thereby avoiding contacting nerves which may cause pain. In such applications, the actual length of the microneedles typically is longer, since the portion of the microneedles distal to the tip may not be inserted into the skin; the uninserted length depends on the particular device design and configuration. The actual (overall) height or length of microneedles should be equal to the insertion depth plus the uninserted length.

The microneedles typically have a gauge size of between 26 Gauge and 31 Gauge, inclusive. Exemplary gauge sizes include 26 Gauge, 27 Gauge, 28 Gauge, 29 Gauge, 30 Gauge, and 31 Gauge.

An array of microneedles can include a mixture of microneedles having different structures, forms, and/or properties, such as length, outer diameter, inner diameter, internal storage volume, cross-sectional shape, spacing between the microneedle, orientation relative to the substrate, material, and release rate. In some forms, the array of microneedles is separated into a plurality of sections, wherein each section contains a single type of microneedles having the same structure, form, and properties.

B. Substrate

The substrate of the microneedle device can be constructed from a variety of materials, including metals, ceramics, semiconductors, organics, polymers, and composites. The substrate includes the base to which the microneedles are attached or integrally formed. In some forms, the substrate is made from the same material as the microneedles, such as those descried above. In some forms, the substrate can be adapted to fit a Luer-Lock syringe or other conventionally used drug delivery device that currently uses hypodermic needles as the barrier penetration method.

In some forms of the microneedle device, the substrate, as well as other components, are formed from flexible materials to allow the microneedle device to fit the contours of the biological barrier, such as the skin, to which the microneedle device is applied. A flexible microneedle device may facilitate more consistent penetration of some biological barriers, because penetration can be limited by deviations in the attachment surface. For example, the surface of human skin is not flat due to dermatoglyphics (i.e., tiny wrinkles) and hair. However, for some biological barriers, a rigid substrate may be preferred.

C. Reservoir

The microneedle device optionally contains one or more reservoir(s) for loading and/or storage of the formulation. In some forms, the reservoir is selectably in connection with the bore of at least one microneedle, such that the reservoir contents can flow from the reservoir and out through the microneedle, into the target tissue. Typically, it is attached to, or integrated into, the substrate, either integrally (as in a one-piece device) or at the moment of drug delivery (as with a Luer-lock type device). The reservoir is to provide suitable, leak-free loading and/or storage of the formulation before it is to be delivered. In some forms, the reservoir can prevent the formulation from contamination and/or degradation. For example, the reservoir can exclude light when the formulation contains photo-sensitive materials, and can include an oxygen barrier material in order to minimize exposure of the formulation to oxygen. In some forms, the reservoir can keep volatile materials inside the reservoir, for example, to prevent water from evaporating, thereby avoiding the formulation to dry out and become undeliverable.

The reservoir can be substantially rigid or readily deformable. The reservoir can be formed from one or more polymers, metals, ceramics, or combinations thereof. In some forms, the reservoir is made from the same material as the substrate, the microneedles, or both.

In some forms, the reservoir includes a volume surrounded by one or more walls, or includes a porous material, such as a sponge, which can retain, for example, the formulation until the material is compressed.

In some forms, the reservoir is formed of an elastic material, such as an elastomeric polymer or rubber. For example, the reservoir can be a balloon-like pouch that is stretched (in tension) when filled with the formulation.

In some forms, the reservoir can include a plurality of compartments that are isolated from one another and/or from a portion of the microneedles in an array. The microneedle device can, for example, be provided to deliver different formulations through different needles, or to deliver the same or different formulations at different rates or at different times. Alternatively, the contents of the different compartments can be combined with one another, for example, by piercing, or otherwise removing, a barrier between the compartments, so as to allow the materials in the compartments to mix. For example, a first compartment contains one or more components of the formulation, while a second compartment contains the rest of the components of the formulation. The formulation can be generated in situ upon combining the contents in the two compartments. In one embodiment, the first compartment contains the carrier of the formulation, while the second compartment contains the therapeutic agent, optionally in a lyophilized powder form.

In some forms, the reservoir is a standard or Luer-Lock syringe adapted to connect to the microneedle array.

D. Additional Features

i. Attachment features

In some forms, the microneedle device includes an adhesive material to secure the microneedle device to the skin, temporarily immobilizing the microneedles while inserted into the skin to deliver the formulation. The adhesive material typically is applied to the substrate (in between the microneedles at their base) or to an attachment collar or tabs adjacent the microneedles.

Care must be taken so that any adhesive material does not plug the bores of hollow microneedles. For example, the adhesive material can be applied in a liquid solution by flooding the top of the substrate below the tips of the microneedles, such as from the side of an array of microneedles, or by using a three-dimensional printing process. The solvent from the liquid solution can then be evaporated, thereby precipitating or gelling the adhesive agent to yield a tacky surface. An alternate method of keeping the tips free of the adhesive material is to choose materials of construction having a hydrophobicity or hydrophilicity to control the wetting of the surface to the microneedle tips.

ii. Multi-cartridge features

A modification of the disposable, single use microneedle device utilizes a reusable triggering device (e.g., a plunger) in combination with a cartridge containing one or more, preferably a plurality, of single-use microneedle devices. For example, the cartridge can be a circular disk having a plurality of microneedle arrays connected to a single-dose reservoir, wherein the cartridge can be loaded into and unloaded from the triggering device. The triggering device can, for example, be designed to move a new dose into position for delivery, compress the reservoir to deliver the formulation, and then eject or immobilize the used array. This type of reusable triggering device also can include a power source, such as a battery, used to operate a built-in measurement device, for example, for analyte measurement of interstitial fluids or electrical verification of needle penetration into skin.

iii. Feedback features

In some forms, the microneedle device includes a feedback means so that the user can (1) determine whether delivery has been initiated; and/or (2) confirm that the reservoir has been emptied, that is delivery complete. Representative feedback means include a sound, a color (change) indicator, or a change in the shape of a deformable reservoir. In another form, the feedback for completion of delivery is simply that the reservoir is pressed flat against the back of the substrate and cannot be further deformed.

The user of the microneedle device typically can determine if the microneedles have been properly inserted into the skin or other tissue through visual or tactile means, that is assessing whether the substrate has been pressed essentially to the tissue surface. For example, if a puddle of the formulation appears near the microneedle device, then the user may infer that the microneedles are not fully inserted, suggesting that the microneedle device needs to be reapplied. The formulation may include a coloring agent to enhance the visual feedback.

In a more complex form, an electrical or chemical measurement is adapted to provide the feedback. For example, penetration can be determined by measuring a change in electrical resistance at the skin or other tissue, or a pH change. Alternately, needle-to-needle electrical resistance can be measured.

E. Exemplary Microneedle Devices

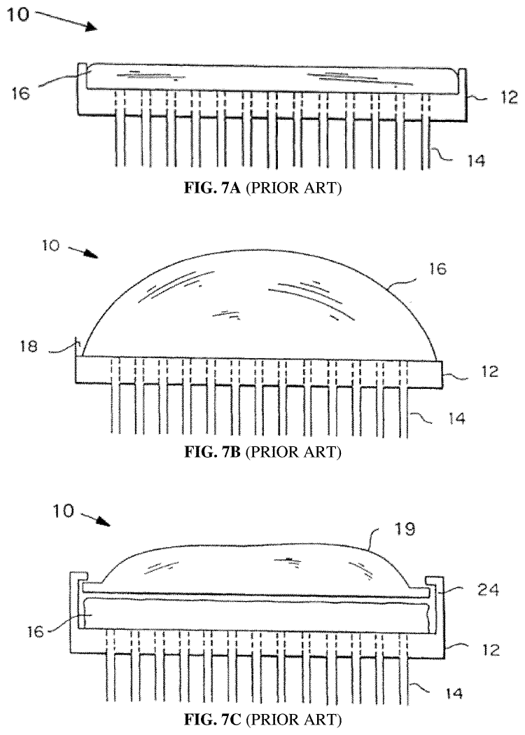

Exemplary microneedle devices can be found in U.S. Pat. No. 6,611,707 and are illustrated in FIGS. 7A-7C. The device 10 includes substrate 12 from which a three-dimensional array of microneedles 14 protrude. As shown, the annular bore of the microneedles 14 extends through the substrate 12. The device 10 also includes a reservoir 16 secured to substrate 12 via a sealing mechanism 18. FIG. 7A shows how the reservoir can be accessed directly by application to the skin, for example, for transdermal delivery of the formulation. The device in FIG. 7B includes a deformable bubble reservoir 16. Manual pressure can be used to expel its contents at the site of application. FIG. 7C shows a separate reservoir 16 from means 19 for expelling the contents of the reservoir 16 at the site of administration. The expelling means 19 can be a flexible bag. The expelling means 19 may also contain a vacuum so that it expands when vented, to create pressure on the reservoir, or it may be elastic so that it deforms when released from one position (not shown). Alternatively, reservoir 16 could be formed of an elastic material which deforms when released.

The sealing mechanism 18 can be, for example, an adhesive material or gasket. The sealing mechanism 18 can further function as or include a fracturable barrier or rate controlling membrane overlaying the surface of the substrate. In this embodiment, nothing can be released until a seal or peel-off strip covering is removed.

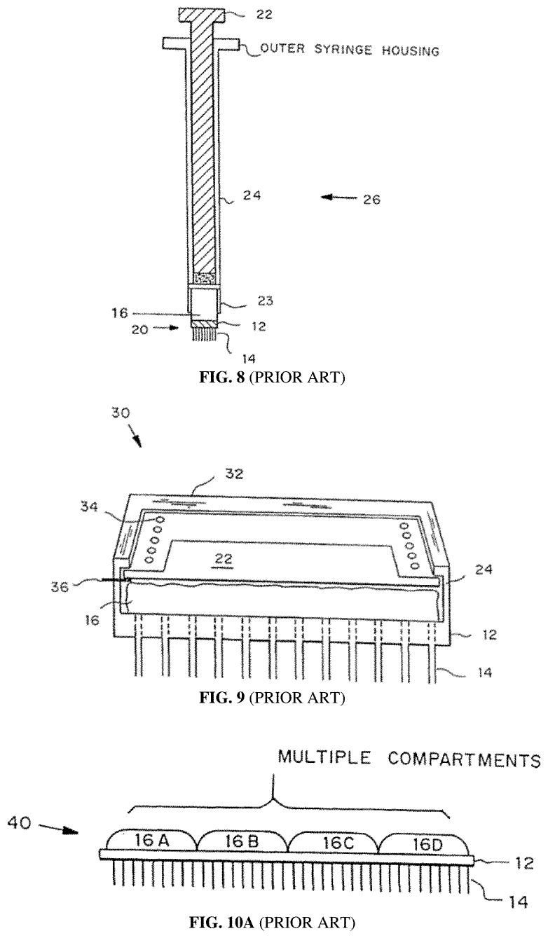

Another exemplary microneedle device is shown in FIG. 8. The device 20 includes substrate 12 from which a three-dimensional array of microneedles 14 protrude. The device 20 also includes plunger 22 that is slidably secured to the upper surface of substrate 12 by plunger guide frame 24 using a restraint such as a Leur-Lock interface 23. The substrate 12 can be attached or detached to a syringe 26 via a connector such as a Luer-Lock type attachment 23. The plunger 22, guide frame 24, and connector 23 connect to, form or contain reservoir 16. A Luer-Lock-type attachment could alternatively be used to secure the device to means for controlling flow or transport through the device such as a pump.

Another exemplary microneedle device is shown in FIG. 9. Like the device in FIG. 8, the device 30 in FIG. 9 includes substrate 12, microneedles 14, plunger 22, plunger guide frame 24, and reservoir 16. Device 30 further includes plunger housing 32, which is attached to, or integrally formed with, plunger guide frame 24. A compressed spring or other tension-based mechanism 34 is positioned between plunger housing 32 and plunger 22. The device 30 further includes spring hold/release mechanism 36, which keeps the plunger up (spring compressed) until triggered to compress reservoir 16.

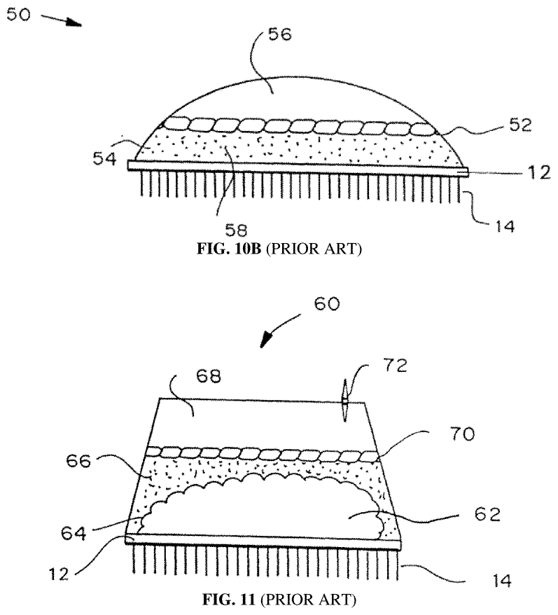

FIG. 10A shows a microneedle device 40 in which microneedles 14 attached to a substrate 12 which is attached to multiple compartments 16a, 16b, 16c, and 16d. Each compartment can contain or function as a reservoir. Material can be expelled from each compartment through all or a subset of microneedles 14.

FIG. 10B depicts a microneedle device 50 in which microneedles 14 are attached to a substrate 12 which is attached to reservoir 58 containing, for example, lyophilized therapeutic agent 54. The reservoir 58 is attached to a fracturable barrier 52 which is attached to another reservoir 56 containing, for example, the pharmaceutically acceptable carrier. If the barrier 52 is fractured, then the two reservoirs 56 and 58 are in fluid communication with each other and their contents can mix. In another embodiment, the reservoir 56 contains a self-emulsifying composition as described above and the reservoir 58 contains an aqueous medium, or vice versa; mixing of the contents from reservoirs 56 and 58 can generate the formulation in situ.

FIG. 11 shows a microneedle device 60 in which microneedles 14 are attached to a substrate 12 which is attached to a reservoir 62. This reservoir is surrounded at least partially by a flexible, impermeable membrane 64. The reservoir is connected to another reservoir 66. The two reservoirs 62 and 66 are separated by the impermeable membrane 64, which is impermeable to the contents of both reservoirs 62 and 66. The reservoir 66 is also connected to another reservoir 68. The two reservoirs 66 and 68 are separated by a rigid, semi-permeable membrane 70, which is partially or completely impermeable.

### V. Methods of Making

A. Making the Formulations

The formulations can be readily prepared using techniques generally known to those skilled in the art. Formation of clear and isotropic microemulsions generally do not require high energy input, such as homogenizers or ultrasound generators. They can be generated even after gentle shaking or mixing.

In certain embodiments, the pharmaceutically acceptable carrier is prepared by mixing the components of the carrier, optionally under stirring. For example, the carrier can be generated by adding water, optionally in a plurality of increments, to the rest of the components of the carrier, under stirring.

In certain embodiments, the therapeutic agent is incorporated into the formulation by mixing it with the carrier, optionally under stirring. In certain embodiments, the therapeutic agent is mixed with the lipophilic compounds or oil first, the mixture of which is then combined with other components to generate the formulations.

For example, the therapeutic agent is first dissolved in the lipophilic compounds or oil. The surfactant(s) and co-surfactant(s) can be incorporated into the lipophilic compounds or oil either prior to or after dissolution of the therapeutic agent. An aqueous medium such as water is then added to the mixture containing the lipophilic compounds or oil, surfactant(s), co-surfactant(s) and therapeutic agent, to generate a clear and isotropic microemulsion. Lastly, a penetration enhancer such as ethanol is optionally incorporated into the microemulsion. Before each step, the corresponding mixture from the last step is optionally stirred, vortexed or gently shaken.

In certain embodiments, the formulations are generated by mixing the corresponding self-emulsifying compositions with an aqueous medium such as water. Optionally, the formulations can be generated in situ by directly administering the corresponding self-emulsifying compositions to a subject in need thereof.

B. Making the Microneedle Devices

The microneedles and substrate can be made by methods known to those skilled in the art. Examples include microfabrication processes, by creating small mechanical structures in silicon, metal, polymer, composites, and other materials. Three-dimensional arrays of hollow microneedles can be fabricated, for example, using combinations of dry etching processes; micromold creation in lithographically-defined polymers and selective sidewall electroplating; or direct micromolding techniques using epoxy mold transfers. These methods are described, for example, in U.S. Pat. Nos. 6,334,856, 6,503,231, 6,611,707, 8,708,966, 10,265,511; in PCT patent application publication WO 2011/076537; Henry, et al., Micro Electro Mechanical Systems, Heidelberg, Germany, 1998, 494-98; Li et al., Curr Med Chem, 2017, 24(22):2413-2422; Cheung et al., Drug Delivery, 2016, 7, 2338-2354; and references cited therein.

### VI. Methods of Using

Administration of the formulations can lead to an improvement or enhancement, of neurological function in a subject with a neurological disease, neurological injury, or age-related neuronal decline or impairment. The neurological disease can be selected from Alzheimer's disease or other neurodegenerative diseases.

Neural deterioration can be the result of any condition which compromises neural function and is likely to lead to neural loss. Neural function can be compromised by, for example, altered biochemistry, physiology, or anatomy of a neuron, including its neurite. Deterioration of a neuron may include membrane, dendritic, or synaptic changes which are detrimental to normal neuronal functioning. The cause of neuron deterioration, impairment, and/or death may be unknown. Alternatively, it may be the result of age-, injury-, and/or disease-related neurological changes which occur in the nervous system of the subject.

Neural loss through disease, age-related decline, or physical insult leads to neuronal decline and impairment. Generally, neural loss implies any neural loss at the cellular level, including loss in neurites, neural organization, and/or neural networks. The formulations disclosed herein can counteract the deleterious effects of neural loss by promoting development of new neurons, new neurites, and/or neural connections, resulting in neuroprotection of existing neural cells, neurites, and/or neural connections. Thus, the neuro-enhancing properties of the formulations can provide an effective strategy to generally reverse the neural loss associated with neurological diseases, aging, and physical injury or trauma.

Methods for treating or preventing neural deterioration or neural loss caused by a neurological disease, neurological injury, or age-related neuronal decline or impairment are provided. The methods include administering an effective amount of a formulation disclosed herein to the subject in need thereof.

As used in this context, an “effective amount” of the formulation refers to an amount that is effective to ameliorate one or more symptoms associated with the neural deterioration or neural loss, including neurological defects or cognitive decline or impairment. Such a therapeutic effect can be generally observed within about 12 to about 24 weeks of initiating administration of the formulation, although the therapeutic effect may be observed in less than 12 weeks or greater than 24 weeks. In certain embodiments, the effective amount of the formulation corresponds to a single dose of between about 4 and about 30 mg of 3a-hydroxy-5a-pregnan-20-one, a derivative or analogue thereof, or a pharmaceutically acceptable salt of the derivative or analogue. Dose regimen (dose and dosing interval) for clinical uses can be estimated, optimized and predicted from preclinical studies and appropriate scaling technique (to predict human dose from animal dose).

Less than 4 mg may not be enough for transdermal administration, since the bioavailability will be less than 100%. Thus, dose needs to be increased to about 15-25 mg if the bioavailability is about 15-20%.

An optimal dosing interval for allopregnanolone is once per week as previous studies showed once per week dosing regimen significantly increased neurogenesis while simultaneously reducing AD pathology (Brinton, Nat Rev Endocrinol, 2013,9(4):241-250; Chen et al., PLoS One, 2011,6(8):e24293; and Irwin et al., Front Endocrinol, 2011,2:117).

The subject in need thereof is preferably an adult human, and more preferably the adult human is over the age of 30, who has lost some amount of neurological function as a result of the neural deterioration or neural loss. Examples of other subjects who can be treated include non-human mammals such as dogs, cats, rats, and mice.

In some embodiments, the methods include repeating the administration weekly or less frequently. For example, a single dose of from about 1 mg to about 30 mg of the therapeutic agent is administered once within a 24-hour period, and the dosing is repeated once a week, or less frequently. In some embodiments, a single dose of from about 1 mg to about 30 mg of the therapeutic agent is administered repeatedly for a total period of one month or longer, such as one month, three months, six months, nine months, one year, or more than one year. In one example, the formulation contains 3a-hydroxy-5a-pregnan-20-one as the therapeutic agent. The formulation is administered to the subject at a single dose of about 24 mg of 3a-hydroxy-5a-pregnan-20-one, repeated once per week or less frequently for a period effective to produce an improvement in at least one criterion set forth as indicative of an improvement in one or more symptoms of the neural deterioration or neural loss.

Suitable improvements include an improvement in cognitive abilities, memory, motor skills, learning or the like. In some embodiments, an improvement is observed in at least two such criteria. Methods for assessing improvement in a particular neurological factor include evaluating cognitive skills, motor skills, memory capacity or the like, as well as assessing physical changes in selected areas of the central nervous system, using magnetic resonance imaging (MRI), computed tomography scans (CT) or other imaging techniques. The methods for such assessments are well known to those skilled in the art, and can be appropriately selected to diagnosis the status of the particular neurological impairment. The assessments can be performed before and after the administration of the formulation for a comparative analysis. Additional assessments can be performed at one or more selected time intervals during the treatment to follow the therapeutic action of the formulation.

The formulation can be administered transdermally or transcutaneously, optionally bypassing the blood brain barrier to the brain. The formulation can be administered using an approach selected from microneedles, intranasal spray, buccal film, transdermal patch, and sublingual capsule or spray. When administration is by way of a transdermal patch, the patch can be applied to deliver a single dose within a 24-hour period. The patch is then removed and another patch is placed on the subject after a period of at least one week, to ensure dosing is not more than once per week. When a single transdermal patch is used to deliver multiple doses, the doses must be separated by a period of time of at least one week to achieve optimal efficacy. Continuous dosing, or dosing more frequently than once per week may lead to neurological decline.

Microemulsions can be applied to various dosing routes including oral, intravenous, transdermal, transcutaneous, topical, nasal, buccal, sublingual, and ocular routes. Oral administration is the most common route for drug delivery. However, oral administration may not be preferred for compounds that are susceptible to chemical degradation in gastrointestinal tract and the first pass metabolism in the liver (Lawrence and Rees, Adv Drug Deliv Rev, 2012, 64:175-193; Vandamme, Prog Retin Eye Res, 2002, 21:15-34; and Heuschkel et al., J Pharm Sci, 2008, 97(2): 603-631). The nasal route can be considered to bypass the blood brain barrier by delivering drugs into the blood cerebrospinal fluid through the olfactory pathway. In addition, the large epithelial surface area and highly vascularized nasal mucosa are beneficial for absorption without the loss of drugs from the first pass metabolism. Microemulsions can be delivered via the nasal route as a spray form (Sintov et al., J Control Release, 2010, 148:168-176; Ilium, J Control Release, 2003, 87:187-198; and Shah et al., Eur J Pharm Sci, 2016, 91:196-207). A main advantage of buccal and sublingual administration is the rapid onset of action as compared to oral administration due to highly vascularized mucosa and drug can be prevented from the first pass metabolism. Microemulsions may be utilized for buccal and sublingual spray, film, and capsule (Sheu et al., J Pharm Sci, 2016, 105:2774-2781; and Padula et al., Eur J Pharm Sci, 2018, 115:233-239). Drug delivery via transdermal, transcutaneous, buccal, sublingual, and ocular routes need to overcome the blood brain barrier once the drugs reach the systemic circulation after administration. The drug penetration across the blood brain barrier depends on multiple factors: (1) characteristics of drugs such as molecular weight and lipophilicity, (2) alteration of the activity of efflux transporters, such as p-glycoprotein, expressed the blood brain barrier, and (3) characteristics of drug carriers such as particle size, shape, and charge (Lu et al., Int J Nanomedicine, 2014, 9:2241-2257; and Marianecci et al., Int J Nanomedicine, 2017, 11:325-335).

A. Treatment of Neurodegenerative Diseases

Neurodegeneration is the progressive loss of structure or function of neurons, including death of neurons. Many neurodegenerative diseases—including amyotrophic lateral sclerosis, Parkinson's disease, Alzheimer's disease, and Huntington's disease—occur as a result of neurodegenerative processes. Such diseases result in progressive degeneration and/or death of neuron cells. Neurodegeneration can be found in many different levels of neuronal circuitry ranging from molecular to systemic.

Alzheimer's disease is an irreversible, progressive neurodegenerative disease. It is characterized by the development of amyloid plaques and neurofibrillary, or tau tangles; the loss of connections between nerve cells (neurons) in the brain; and the death of these nerve cells. There are two types of Alzheimer's—early-onset and late-onset. Both types have a genetic component. Early-onset Alzheimer's disease occurs between a person's 30s to mid-60s and represents less than 10 percent of all people with Alzheimer's disease. Some cases are caused by an inherited change in one of three genes, resulting in a type known as early-onset familial Alzheimer's disease, or FAD. For other cases of early-onset Alzheimer's disease, research suggests there may be a genetic component related to factors other than these three genes. Most people with Alzheimer's have the late-onset form of the disease, in which symptoms become apparent in the mid-60s and later. The causes of late-onset Alzheimer's are not yet completely understood, but they likely include a combination of genetic, environmental, and lifestyle factors that affect a person's risk for developing the disease.

In Alzheimer's patients, neural loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory.

Methods for treating or preventing neuronal damage and/or the associated cognitive decline or impairment, caused by Alzheimer's disease or other neurodegenerative diseases, include administering an effective amount of a formulation disclosed herein to a subject in need thereof. In certain embodiments, the methods can be used to reduce, prevent, or reverse the learning and/or memory deficits in the subject suffering from Alzheimer's disease and/or other neurodegenerative diseases. Neuro-enhancement resulting from the administration of the formulation includes the stimulation or induction of neural mitosis leading to the generation of new neurons, i.e., exhibiting a neurogenic effect, prevention or retardation of neural loss, including a decrease in the rate of neural loss, i.e., exhibiting a neuroprotective effect, or one or more of these modes of action. The term “neuroprotective effect” is intended to include prevention, retardation, and/or termination of deterioration, impairment, or death of the subject's neurons, neurites, and/or neural networks.

The clinical symptoms of Alzheimer's disease and/or other neurodegenerative diseases include cognitive disorders such as dementia. For example, the clinical symptoms of Alzheimer's disease include those of mild Alzheimer's disease, moderate Alzheimer's disease, and/or sever Alzheimer's disease.

In mild Alzheimer's disease, a person may seem to be healthy but has more and more trouble making sense of the world around him or her. The realization that something is wrong often comes gradually to the person and their family. Exemplary symptoms of mild Alzheimer's disease include, but are not limited to: memory loss, poor judgment leading to bad decisions, loss of spontaneity and sense of initiative, taking longer to complete normal daily tasks, repeating questions, having trouble handling money and paying bills, wandering and getting lost, losing things or misplacing them in odd places, mood and personality changes, and increased anxiety and/or aggression.

Symptoms of moderate Alzheimer's disease include, but are not limited to: forgetfulness, increased memory loss and confusion, inability to learn new things, difficulty with language and problems with reading, writing, and working with numbers, difficulty organizing thoughts and thinking logically, shortened attention span, problems coping with new situations, difficulty carrying out multistep tasks, such as getting dressed, problems recognizing family and friends, hallucinations, delusions, paranoia, impulsive behavior such as undressing at inappropriate times or places or using vulgar language, inappropriate outbursts of anger, restlessness, agitation, anxiety, tearfulness, wandering (especially in the late afternoon or evening), repetitive statements or movement, and occasional muscle twitches.

Symptoms of severe Alzheimer's disease include, but are not limited to: inability to communicate, weight loss, seizures, skin infections, difficulty swallowing, groaning, moaning, grunting, increased sleeping, and loss of bowel and bladder control.

The clinical symptoms of Alzheimer's disease and/or other neurodegenerative diseases also include physiological symptoms, such as reduction in brain mass, for example, reduction in hippocampal volume. Therefore, in some embodiments, administering the formulation can increase the hippocampal volume of the subject or reduce or prevent the rate of decrease of hippocampal volume, as compared to an untreated control subject or the same subject prior to the administration of the formulation.

Administration of the same dosage of the formulation, preferably given once, so that the active agent is delivered completely within a period of time of less than two hours, is administered again to the same subject after a period of at least 7 days, after 8 days, after 9 days, or after more than 9 days, in cycles of no more frequently than once per week. In some embodiments, a dosage regimen “cycle” includes administering a first dose of an amount of 3a-hydroxy-5a-pregnan-20-one between about 4 and about 30 mg on day 1, then no dose on day 2, no dose on day 3, no dose on day 4, no dose on day 5, no dose on day 6, no dose on day 7. A second cycle includes administering a second dose of the formulation between about 4 and about 30 mg on day 8, then no dose on day 9, no dose on day 10, no dose on day 11, no dose on day 12, no dose on day 13, and no dose on day 14. This regimen is repeated for as many cycles as is deemed effective to treat one or more symptoms of Alzheimer's disease and/or other neurodegenerative diseases, or to prevent or delay the onset of one or more symptoms of Alzheimer's disease and/or other neurodegenerative diseases. For example, the formulation can be administered a total of 5-10 times over about 10 weeks, a total of about 15-30 times over about 30 weeks, a total of 30-60 times over about 60 weeks, etc. Preferably, the formulation is administered regularly once per week or less frequently for as long as the subject is receiving noticeable benefit from the treatment method.

An exemplary dosing interval for formulations containing 3a-hydroxy-5a-pregnan-20-one (allopregnanolone) is once per week as previous studies showed such a dosing interval can significantly increase neurogenesis while simultaneously reducing Alzheimer's disease pathology (see Brinton, Nat Rev Endocrinol, 2013, 9(4):241-250; Chen et al., PLoS ONE, 2011, 6(8):e24293; and Irwin et al., Front Endocrinol, 2011, 2:117).

B. Routes of Administration

In preferred embodiments, the formulation is administered via a topical route, optionally bypassing the blood brain barrier to the brain. The formulation can be administered using an approach selected from microneedles, intranasal spray, buccal film, capsule or spray, transdermal patch, and sublingual film, capsule or spray.

Nasal administration can be considered to bypass the blood brain barrier by delivering drugs into the cerebrospinal fluid through the olfactory pathway. In addition, the large epithelial surface area and highly vascularized nasal mucosa are beneficial for absorption without the loss of drugs from the first pass metabolism. The formulation can be delivered via the nasal route as a spray form (Sintov et al., J Control Release, 2010, 148:168-176; Illum, J Control Release, 2003, 87:187-198; and Shah et al., Eur J Pharm Sci, 2016, 91:196-207).

The formulation may be administered via buccal or sublingual film, capsule or spray (Sheu et al., J Pharm Sci, 2016, 105:2774-2781; and Padula et al., Eur J Pharm Sci, 2018, 115:233-239).

When administration is by way of a transdermal patch, the patch can be applied to deliver a single dose within a 24-hour period. The patch is then removed and another patch is placed on the subject after a period of at least one week, to ensure dosing is not more than once per week. When a single transdermal patch is used to deliver multiple doses, the doses must be separated by a period of time of at least one week to achieve optimal efficacy. Continuous dosing, or dosing more frequently than once per week may lead to neurological decline.

In certain embodiments, the formulation is administrated via a microneedle device to a subject in need thereof. The administration can be performed by applying the microneedle device to the skin of the subject. In some forms, delivery of the formulation from the microneedle device is initiated by applying a force, such as by pressing the top of the reservoir, to cause the formulation to flow out through the microneedles, an active or dynamic process. For example, the user can apply finger-pressure directly to a deformable reservoir “bubble,” or to a plunger mechanism as illustrated in U.S. Pat. No. 6,611,707. In some forms, the force ruptures a fracturable barrier between the reservoir contents and the inlet of the microneedle. Representative barriers include thin foil, polymer, or laminant films. In some forms, the microneedles tips are blocked until immediately before use. The blocking material can be, for example, a peelable adhesive or gel film, which will not clog the openings in the microneedle tip when the film is removed from the microneedle device.

In some forms, delivery is initiated by opening the pathway between the reservoir and the microneedle tip, or unblocking the tip openings, and simply allowing the therapeutic agent to be delivered by diffusion, that is, a passive process. For example, delivery can be initiated by opening a mechanical gate or valve interposed between the reservoir outlet and the microneedle inlet.

In some forms, the microneedles become cracked or shattered during and/or after being inserted into the biological barrier, thereby immediately releasing the encapsulated formulation into the target site.

The microneedle device can be capable of transporting the formulation or therapeutic agent across or into the tissue at a useful rate. The rate of delivery of the formulation or therapeutic agent can be controlled by altering one or more of several design variables. For example, the amount of the formulation or therapeutic agent flowing through the needles can be controlled by manipulating the effective hydrodynamic conductivity (the volumetric through-capacity) of the microneedle device, for example, by using more or fewer microneedles, by increasing or decreasing the number or diameter of the bores in the microneedles, or by filling at least some of the microneedle bores with a diffusion-limiting material. It is preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the formulation or therapeutic agent. For example, in passive diffusion systems, the concentration of the therapeutic agent in the formulation can be increased to increase the rate of mass transfer. In active systems, for example, the pressure applied to the reservoir can be varied.

### VII. EXAMPLES

### Example 1.

### Solubility of Allo in oils

Methods

The solubility of Allo was determined in CAPMUL® MCM EP/NF (monodiglyceride of medium chain fatty acids, commercially available from Abitec Corporation (Columbus, Ohio), CAS Number 91744-32-0, or 26402-22-2, and 26402-26-6), isopropyl myristate, and oleic acid, by adding an excess amount of Allo to 1 mL of each oil in a glass vial. The vial was rocked for 72 h at room temperature. The excessive amount of Allo was filtered through a syringe membrane filter (0.2 μm). The amount of Allo dissolved in each oil was determined using HPLC.

An HPLC analytical method was developed by employing the SHIMADZU LC₂₀₁₀A HT for in vitro and ex vivo evaluation of Allo from microemulsions (MEs). Chromatographic separation was achieved using a PHENOMENEX® KINETEX® Phenyl-hexyl 2.6 μm reverse-phase (150×4.6 mm) column. The mobile phase was a mixture of 0.1% acetic acid in water and methanol, 20:80 (v/v) and flowed at 0.4 ml/min for 15 min The injection volume was 10 μl and the UV detection wavelength was 206 nm. A standard curve was constructed at the concentration range of 7.8-1,000

Results

The saturated solubilities of Allo in the tested oils were 28.35±1.92, 8.88±0.17, and 17.8±3.97 mg/ml in CAPMUL® MCM, isopropyl myristate, and oleic acid, respectively, as shown in Table 1. Since Allo showed the highest solubility in CAPMUL® MCM, it was selected as an oil phase to establish pseudo ternary phase diagrams and to optimize the compositions of Allo MEs.

TABLE 1
Saturated solubility of Allo in oils.

| | Solubility of Allo |
|---|---|
| Oil | (mg/ml, mean± SD, n =3) |
| CAPMUL ® MCM | 28.35 ± 1.92 |
| Isopropyl myristate | 8.88 ± 0.17 |
| Oleic acid | 17.8 ± 3.97 |

### Example 2

### Construction of Pseudo Ternary Phase Diagrams

Methods

MEs are thermodynamically stable and isotropic mixtures of oil, water, and surfactant(s)/co-surfactant(s). To develop Allo MEs, oils and surfactants commonly employed in MEs were initially screened by combining these components at a 50:50 weight ratio. Tested oils were CAPTEX® 300 EP/NF (medium chain triglycerides, CAS Number 65381-09-1), CAPMUL® MCM EP/NF (CAS Number 91744-32-0, or 26402-22-2, and 26402-26-6), isopropyl myristate, and oleic acid. Surfactants/co-surfactants tested were TWEEN® 80 (polysorbate 80), CREMOPHOR EL® (PEG-35 castor oil, CAS number 61791-12-6), SPAN® 80 (sorbitan monooleate), LABRAFIL® M 1944 CS (oleoyl polyoxyl-6-glycerides) and TRANSCUTOL® P (diethylene glycol monoethyl ether). Ethanol, propylene glycol, and polyethylene glycol 400 were tested as solvents, and ethanol and propylene glycol were further evaluated as a penetration enhancer in the in vitro permeation study. The amount of water incorporated in the mixture of oil and surfactants was determined by adding water in 0.1 g increments until the mixtures changed from transparent to turbid. The promising combinations of oils and surfactants were further screened by combining these components at different weight ratios, 10:90, 30:70, 50:50, 70:30, and 90:10. Based on the maximum solubility of Allo in oils, miscibility of each component, and water percent in MEs, CAPMUL® MCM was selected as the oil phase, TWEEN® 80 and SPAN® 80 were selected as the surfactants, and TRANSCUTOL® P was selected as the co-surfactant. Pseudo ternary phase diagrams were constructed to determine the regions of MEs by combining CAPMUL® MCM with mixtures of TWEEN® 80 and TRANSCUTOL® P, SPAN® 80 and TRANSCUTOL® P, or TWEEN® 80, SPAN® 80 and TRANSCUTOL® P at different weight ratios, including 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, and 5:95. Water was added dropwise until the mixtures changed from transparent to turbid. The percentage of each component that changed the transparency of the mixtures was pointed and connected in the diagrams, and then the region of MEs was determined.

Results

MEs are metastable colloidal systems comprised of droplets of one liquid dispersed within another immiscible liquid with the presence of emulsifying agents or surfactants (Callender et al., International Journal of Pharmaceutics, 2017, 526(1-2):425-42). Based on the miscibility of various oils and surfactants tested, TWEEN® 80 (polysorbate 80) and SPAN® 80 (sorbitan monooleate) were selected as the surfactants for further studies in the development process of Allo MEs. Nonionic surfactants, such as TWEEN® 80 and SPAN® 80, are less irritating to the skin and less toxic compared to other types of surfactants (Kovacevic et al., International Journal of Pharmaceutics, 2011, 406(1-2):163-72; Effendy and Maibach, Contact Dermatitis, 1995, 33(4):217-25). TRANSCUTOL® P (diethylene glycol monoethyl ether) was selected as the co-surfactant of Allo MEs, which is commercially available from Gattefossé (Lyon, France). It has strong solubilizing characteristics with low toxicity and has a long history of safe use in many products including pharmaceuticals, cosmetics and food applications (Sullivan Jr. et al., Food and Chemical Toxicology, 2014, 72:40-50). TRANSCUTOL® P and TWEEN® 80 are also known as penetration enhancers (Lane, International Journal of Pharmaceutics, 2013, 447(1-2):12-21).

The constructed pseudo ternary phase diagrams shown in FIGS. 1A-1C indicated the regions of mono-phase and stable MEs when mixing oil, surfactant(s), co-surfactant, and water. The compositions of MEs to construct the diagrams are CAPMUL® MCM:[SPAN® 80:TRANSCUTOL® P, 1:1, w/w]:water for the diagram in FIG. 1A, CAPMUL® MCM:[TWEEN® 80:TRANSCUTOL® P, 1:1, w/w]:water for the diagram in FIG. 1B, and CAPMUL® MCM:[TWEEN® 80:SPAN® 80:TRANSCUTOL® P, 1:1:8, w/w/w]:water for the diagram in FIG. 1C. Based on the established pseudo ternary phase diagrams, the weight percent ranges of CAPMUL® MCM, TWEEN® 80, SPAN® 80, TRANSCUTOL® P, ethanol (penetration enhancer), and water were determined within the maximum percent of each inactive ingredient (IIG) in the FDA IIG database or within minimum ranges that construct MEs (Tables 2, 3, and 4). Ethanol, known as a penetration enhancer (Lane, International Journal of Pharmaceutics, 2013, 447(1-2):12-21; Williams and Barry, Advanced Drug Delivery Reviews, 2012, 64:128-37; Verma and Fahr, Journal of Controlled Release, 2004, 97(1):55-66), was included in the MEs to enhance the permeation of Allo across target membranes (e.g., skin, nasal, buccal, sublingual, etc.).

TABLE 2
Weight percent ranges of each component of MEs in the pseudo
ternary phase diagram shown in FIG. 1A.

| | | Surfactant/co-surfactant | |
|---|---|---|---|
| Oil | | (SPAN ® 80:TRANSCUTOL ® P, | |
| (CAPMUL ® | | w/w) and penetration enhancer (ethanol) | |
| MCM) | | *Up to 20 wt % of ethanol in the MEs | Water |
| 7-7.9 | wt % | 85-88 wt % (1:1, w/w) with or without | 5-8 wt % |
| | | ethanol | |
| 8-8.9 | wt % | 82-88 wt % (1:1, w/w) with or without | 4-10 wt % |
| | | ethanol | |
| 9-9.9 | wt % | 81-87 wt % (1:1, w/w) with or without | 4-10 wt % |
| | | ethanol | |
| 10-10.9 | wt % | 79-86 wt % (1:1, w/w) with or without | 4-11 wt % |
| | | ethanol | |
| 11-11.9 | wt % | 77-85 wt % (1:1, w/w) with or without | 4-12 wt % |
| | | ethanol | |
| 12-13 | wt % | 73-84 wt % (1:1, w/w) with or without | 4-14 wt % |
| | | ethanol | |

TABLE 3
Weight percent ranges of each component of MEs in the pseudo
ternary phase diagram shown in FIG. 1B.

| | | | Water and | |
|---|---|---|---|---|
| | | | penetration | |
| | | | enhancer | |
| | | | (ethanol) | |
| | | | *Up to | |
| Oil | | Surfactant/co-surfactant | 20 wt % | |
| (CAPMUL ® | | (TWEEN ® 80:TRANSCUTOL ® | of ethanol | |
| MCM) | | P, w/w) | in the MEs | |
| 0.01-1.5 | wt % | 12-30 wt % (1:1, w/w) | 68.5-87.99 | wt % |
| 1.6-2 | wt % | 13-30 wt % (1:1, w/w) | 68-85.4 | wt % |
| 2.1-3 | wt % | 16-30 wt % (1:1, w/w) | 67-81.9 | wt % |
| 3.1-13 | wt % | 27-30 wt % (1:1, w/w) | 57-69.9 | wt % |

TABLE 4
Weight percent ranges of each component of MEs in the pseudo
ternary phase diagram shown in FIG. 1C.

| | Surfactants/co-surfactant/penetration | |
|---|---|---|
| | enhancer (TWEEN ® 80:SPAN ® | |
| Oil | 80:TRANSCUTOL ® P:ethanol, | |
| (CAPMUL ® MCM) | w/w/w/w) | Water |
| 7-12 wt % | 87 wt % (1:1:5.7:2.3, w/w/w/w) | 1-6 wt % |
| 8-13 wt % | 85 wt % (1:1:5.65:2.35, w/w/w/w) | 2-7 wt % |
| 10-13 wt % | 83 wt % (1:1:5.59:2.41, w/w/w/w) | 4-7 wt % |
| 11-13 wt % | 82 wt % (1:1:5.56:2.44, w/w/w/w) | 5-7 wt % |
| 12-13 wt % | 81 wt % (1:1:5.53:2.47, w/w/w/w) | 6-7 wt % |
| 12-13 wt % | 81 wt % (1:1:6.15:1.85, w/w/w/w) | 6-7 wt % |

### Example 3

### In Vitro Cell Viability Study After the Treatment of Allo

Methods

Cell viability was evaluated in human skin (HaCaT)cell line by performing a resazurin assay after the treatment of Allo. Resazurin is a cell permeable redox indicator, and viable cells with active metabolism can reduce resazurin into resorufin, which is fluorescent (Riss et al., Cell Viability Assays, in Assay Guidance Manual, 2016). The HaCaT cells were grown in optimized Dubelco's Modified Eagle's medium (DMEM) (containing 2 mM of L-glutamine, 2 mM of sodium pyruvate, 4500 mg/L of glucose, and 1500 mg/L of sodium bicarbonate) supplemented with 10% fetal bovine serum (FBS). The TR 146 cells were grown in Ham's F12 medium supplemented with 10% FBS, 1% penicillin-streptomycin, and 0.2% L-Glutamine The RPMI 2650 cells were grown in Eagle's Minimum Essential medium (EMEM) supplemented with 10% FBS, 1% penicillin-streptomycin, and 0.2% amphotericin D. Cells were plated in a 96-well plate at a density of 5,000 cells per each well (100 μl) and incubated at 37° C. and 5% CO2 for 48 h. Allo was prepared in cell culture media with the addition of 1% (v/v) ethanol to aid Allo solubility. The final concentration range of Allo treated to the cells was 0.001-10 μM. One hundred μl (100 μl) of the Allo solutions was added to each well (n=6 for each concentration) and the 96-well plate was incubated at 37° C. and 5% CO₂ for 72 h. The control group was treated with a blank solution (i.e., 1% (v/v) ethanol in the corresponding cell culture medium) without Allo. After 72 h, 20 μl of a resazurin solution (20 μM) was added to each well and the 96-well plate was incubated at 37° C. and 5% CO₂ for 3 h. The cell viability was measured using Synergy H1 Multi-Mode Reader (BioTek Instruments Inc., Winooski, Vt.) at the excitation wavelength of 544 nm and emission wavelength of 590 nm.

Results

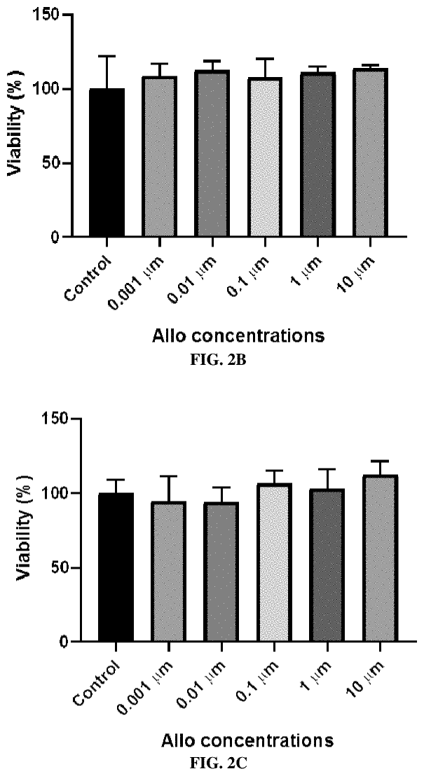

The viability of human skin cell line was investigated after the treatment of Allo for 72 h. The viability percent ranges were 84.16-102.70% in the HaCaT (human skin) cell line, 93.89-112.44% in the TR 146 (human buccal) cell line, and 107.32-113.60% in the RPMI 2650 (human nasal) cell line (FIGS. 2A-2C). No significant decrease in viability was observed compared to that in the control group that did not contain Allo.

### Example 4

### Solubility of Allo in MEs

Methods

The saturated solubilities of Allo in three lead MEs were determined. The compositions of the lead MEs were as follows:
(1) ME-A=CAPMUL® MCM:SPAN® 80:TRANSCUTOL® P:water:ethanol, 8:34:34:4:20, by wt %;
(2) ME-B=CAPMUL® MCM:TWEEN® 80:TRANSCUTOL® P:water:ethanol, 13:15:15:42:15, by wt %; and
(3) ME-C=CAPMUL® MCM:TWEEN® 80:SPAN® 80:TRANSCUTOL® P:water:ethanol, 13:8.1:8.1:49.8:6:15, by wt %.

An excess amount of Allo was added into the MEs. The mixture was rocked for 72 h at room temperature. The excessive amount of Allo was filtered through a syringe membrane filter (0.2 μm) and the filtered solution was injected into HPLC for analysis after dilution with ethanol.

Results

Three lead MEs were selected based on the minimum percent of each inactive ingredient that needs to form MEs, and the saturated solubilities of

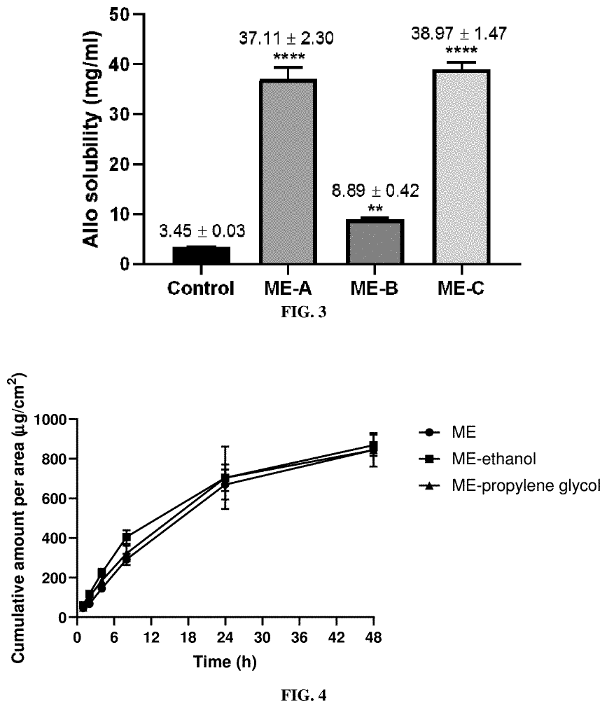

Allo in the selected MEs were determined as presented in FIG. 3. The solubilities were 5.93-25.98-fold increased as compared to Allo in 0.9% sodium chloride with 6% sulfobutyl-ether-β-cyclodextrin solution (DEXOLVE™) prepared at 1.5 mg/ml for its intravenous/intramuscular administration in the phase 1 clinical trials. The highest solubility, 38.97±1.47 mg/ml, was shown in the ME-C formulation, i.e., CAPMUL® MCM:TWEEN® 80:SPAN® 80:TRANSCUTOL® P:water:ethanol, 13:8.1:8.1:49.8:6:15 (by wt %), which was 25.98-fold increased as compared to the intravenous/intramuscular solution. The solubilities of Allo were 24.74-fold increased (37.11±2.30 mg/ml) in the ME-A formulation, i.e., CAPMUL® MCM:SPAN® 80:TRANSCUTOL® P:water:ethanol, 8:34:34:4:20 (by wt %) and 5.93-fold increased (8.89±0.42 mg/ml) in the ME-B formulation, i.e., CAPMUL® MCM:TWEEN® 80:TRANSCUTOL® P:water:ethanol, 13:15:15:42:15 (by wt %), as compared to the intravenous/intramuscular solution.

### Example 5

### Characterization of MEs: Viscosity and Morphology of Droplets

Methods

The viscosity of Allo-unloaded MEs was measured using an Ostwald viscometer at room temperature (24±1° C.). The viscosity was calculated by the following equation:

$$η x = η w \frac{dxTx}{dwTw}$$

wherein ηx is the viscosity of MEs; ηx is the viscosity of water; dx is the density of MEs; dw is the density of water; Tx is the time of flow of MEs; and Tw is the time of flow of water.

Transmission electron microscopy (TEM, TECNAI G2) was utilized to investigate the morphology and size of droplets in Allo MEs at 100 kV voltage. Allo MEs were negatively stained by 1% phosphotungstic acid (PTA) solution and dried before measurement.

Results It is known that viscosity may influence the drug release from MEs and increasing the viscosity of emulsions generally causes a more rigid structure (Tsai et al., Journal of Pharmaceutical Sciences, 2011, 100(6):2358-65). The viscosity was measured in the following compositions of the MEs: (1) CAPMUL® MCM:SPAN® 80:TRANSCUTOL® P:water:ethanol, 8:34:34:4:20, by wt % (ME-A), (2) CAPMUL® MCM:TWEEN® 80:TRANSCUTOL® P:water:ethanol, 13:15:15:42:15, by wt % (ME-B), and (3) CAPMUL® MCM:TWEEN® 80:SPAN® 80:TRANSCUTOL® P:water:ethanol, 13:8.1:8.1:49.8:6:15, by wt % (ME-C). The viscosities of ME-A, ME-B, and ME-C (Allo-unloaded) were determined as 6.79±0.03, 9.96±0.05, and 5.40±0.01 cP (mean±SD, n=6), respectively (Table 5). The droplet size ranges of Allo ME-A, ME-B, and ME-C estimated by TEM were 37.56-125.5, 16.2-79.96, and 31.29-122.4 nm, respectively.

TABLE 5
Viscosity of MEs (Allo-unloaded).

| | | Viscosity |
|---|---|---|
| ME | Composition (wt %) | (cP) |
| ME-A | CAPMUL ® MCM:SPAN ® 80:TRANSCUTOL ® | 6.79 ± 0.03 |
| | P:water:ethanol = 8:34:34:4:20 (wt %) | |
| ME-B | CAPMUL ® MCM:TWEEN ® | |
| | 80:TRANSCUTOL ® P:water:ethanol = | 9.96 ± 0.05 |
| | 13:15:15:42:15 (wt %) | |
| ME-C | CAPMUL ® MCM:TWEEN ® 80:SPAN ® | 5.40 ± 0.01 |
| | 80:TRANSCUTOL ® P:water:ethanol = | |
| | 13:8.1:8.1:49.8:6:15 (wt %) | |

### Example 6

### Stability of Allo MEs

Methods

The stabilities of Allo MEs were evaluated for one month at the accelerated condition (40° C. and 75% relative humidity) as described in the FDA guidance (Group IEW, editor Q1A (R2), Stability Testing of New Drug Substances and Products. International Conference on Harmonisation of Technical Requirements for the Registration of Pharmaceuticals for Human Use Geneva: ICH; 2003). The concentration and weight of Allo MEs after the storage condition and period were determined and compared with those measured on the day of preparation. Phase separation, color, transparency, and Allo precipitation were visually evaluated. The compositions of Allo MEs for the stability testing were as follows:
(1) ME-A=CAPMUL® MCM:SPAN® 80:TRANSCUTOL® P:water:ethanol, 8:34:34:4:20, by wt %; and
(2) ME-B=CAPMUL® MCM:TWEEN® 80:TRANSCUTOL® P:water:ethanol, 13:15:15:42:15, by wt %.

Results

Allo was stable in the MEs at 40° C. and 75±5% relative humidity for one month (Table 6). There were no significant differences in Allo concentrations and weight of ME-A and ME-B measured on the day of preparation and after one month. Phase separation, Allo precipitation, color changes, and transparency changes were not observed.

TABLE 6
Stability of Allo MEs at the accelerated condition at 40° C. and
75 ± 5% relative humidity for one month.

| | | Allo concentration | | | |
|---|---|---|---|---|---|
| | | (mg/g) | | Weight of MEs (g) | |
| ME | Composition (wt %) | Day 0 | 1 month | Day 0 | 1 month |
| ME-A | CAPMUL ® MCM:SPAN ® | 30.31 ± 2.98 | 30.34 ± 1.03 | 0.92 ± 0.004 | 0.91 ± 0.06 |
| | 80:TRANSCUTOL ® | | | | |
| | P:water:ethanol, 8:34:34:4:20, | | | | |
| | by wt % | | | | |
| ME-B | CAPMUL ® MCM:TWEEN ® | 6.11 ± 0.03 | 6.15 ± 0.01 | 0.95 ± 0.001 | 0.93 ± 0.01 |
| | 80:TRANSCUTOL ® | | | | |
| | P:water:ethanol, 13:15:15:42:15, | | | | |
| | by wt % | | | | |

### Example 7

### Selection of a Receptor Medium for the In Vitro Permeation Study

Methods

To select a receptor medium placed in the receptor compartment of the Franz chamber, the saturated solubilities of Allo in various media were determined. PBS at pH 7.4 has been commonly used to mimic the physiological condition. However, Allo (log P of 5.042) has a limited solubility in aqueous phase. Hence, low percent of solubilizers was added to PBS (pH 7.4) to enhance Allo solubility in the receptor medium and to maintain the sink condition during the in vitro permeation study. An excessive amount of Allo was added to 1 ml of the following media: (1) 10% (w/v) 2-hydroxypropyl-β-cyclodextrin (HβCD), (2) 40% (v/v) isopropyl alcohol (IPA), (3) 30% (v/v) CREMOPHOR EL®, and (4) 20% (v/v) IPA and 25% (v/v) CREMOPHOR EL®. The solutions were kept under constant magnetic stirring at 600 rpm for 24 h and placed in an incubator at 32° C. After 24 h, the excessive amount of Allo was filtered through a 0.2 μm syringe membrane and the filtered solution was injected into the HPLC for analysis.

Results

Table 7 shows the solubility of Allo in different receptor media. PBS (pH 7.4) containing 10% (w/v) of HβCD was selected for the receptor medium to maintain sink condition in in vitro permeation studies, since Allo solubility was the highest in the HβCD solution (3.45±0.03 mg/ml) among the tested media, and HβCD does not affect drug permeation. Based on the U.S. Pharmacopeia (general chapter 1092), the sink condition is defined as the volume of medium at least three times that required in order to form a saturated solution of drug substance (Formulary USPN, General Chapter <1092>, The Dissolution Procedure: Development and Validation, 2014). In the subsequent permeation study, a dose placed in the donor compartment was ˜1 mg and the volume of the receptor medium was ˜4 ml. The maximum concentration that can be attained in the receptor compartment was 0.25 mg/ml, which was 7.25% of the saturated Allo concentration of the selected receptor medium. Thus, the sink condition can be maintained during the subsequent permeation study.

TABLE 7
Solubility of Allo in different receptor media.

| | Solubility (mg/ml), |
|---|---|
| Media | mean ± SD, n = 3 |
| PBS (pH 7.4) containing: | |
| 10% (w/v) HβCD | 3.45 ± 0.03 |
| 40% (v/v) IPA | 1.13 ± 0.13 |
| 30% (v/v) CREMOPHOR EL ® | 1.99 ± 0.17 |
| 20% (v/v) IPA and 25% (v/v) | 3.35 ± 0.12 |
| CREMOPHOR EL ® | |

### Example 8

### Permeation of Allo Through Skin Membrane

Methods

To evaluate the skin permeation of Allo from MEs, the Franz diffusion cell system was utilized. The STRAT-M® non-animal based, synthetic membrane with a thickness of 300 μm was selected for the evaluation of Allo permeation across the skin. The dimensions of the Franz cell were 0.95 cm² for the top surface area and ˜4 ml for the receiver volume. The membrane was mounted at the interface between the donor and receptor compartments. The receptor medium was 10% (w/v) HβCD in PBS (pH 7.4) and stirred at 600 rpm. The Franz cell was maintained at 32° C. to mimic skin temperature. One mg (1 mg) of Allo was placed into the donor compartment and 500 μl of samples was collected from the receptor compartment at 0.5, 1, 2, 4, 8, 24, and 48 h post dose. The same volume of the receptor medium was replenished after sampling. The samples were centrifuged at 14,000 rpm and 25° C. for 15 min and injected into HPLC for analysis. Flux and permeability coefficients were calculated based on a slope before the curve of the cumulative amount versus the time profile reaches the plateau.

Results

The purposes of in vitro permeation studies were to investigate the effect of penetration enhancers on the permeation of Allo across skin membrane and to evaluate in vitro transdermal permeation profiles of Allo MEs.

Penetration enhancers tested were ethanol, propylene glycol, and glycerol. Since glycerol was not well-miscible with Allo-MEs, ethanol and propylene glycol were further evaluated in the in vitro permeation studies. The effect of penetration enhancers was evaluated by adding 20 wt % of ethanol or propylene glycol to a ME composition, i.e., CAPMUL® MCM:SPAN® 80:TRANSCUTOL® P:water at weight ratios of 10:42.5:42.5:5. Table 8 summarizes the ME compositions with or without the penetration enhancers. The cumulative amounts of Allo permeated across the membrane were 845.36±83.99 μg/cm² without adding penetration enhancers, and 869.13±53.52 μg/cm² by adding 20 wt % of ethanol (FIG. 4). The cumulative amounts of Allo were comparable without (845.36±83.99 μg/cm²) or with the addition of 20 wt % of propylene glycol (844.70±8.49 μg/cm²). The percent release of Allo across the membrane for 48 h was 80.31±7.98% without adding the penetration enhancers, 82.57±5.08% by adding ethanol, and 80.25±0.81% by adding propylene glycol. The flux and permeability coefficient of Allo was significantly increased when ethanol is added to the ME (Table 9). However, there was no significant effect of propylene glycol on the flux and permeability coefficient of Allo. Hence, ethanol was selected as the penetration enhancer and incorporated into lead Allo MEs.

TABLE 8
ME compositions with or without the penetration enhancers.

| | Composition (wt %) | | | | | |
|---|---|---|---|---|---|---|
| | CAPMUL ® | SPAN ® | TRANSCUTOL ® | | | Propylene |
| | MCM | 80 | P | Water | Ethanol | glycol |
| ME | 10 | 42.5 | 42.5 | 5 | 0 | 0 |
| ME-ethanol | 8 | 34 | 34 | 4 | 20 | 0 |
| ME-propylene | 8 | 34 | 34 | 4 | 0 | 20 |
| glycol | | | | | | |

TABLE 9
Effect of penetration enhancers on flux and permeability
coefficients of Allo. Each data point represents mean ± SD. *P < 0.05,
compared to the ME group.

| | | Flux | Permeability coefficient |
|---|---|---|---|
| | n | (μg/cm²/h) | (×10⁻³ cm/h) |
| ME | 5 | 37.21 ± 4.41 | 3.72 ± 0.44 |
| ME-ethanol | 3 | 54.18 ± 5.52* | 5.42 ± 0.55* |
| ME-propylene | 3 | 44.51 ± 10.22 | 4.45 ± 1.02 |
| glycol | | | |

The permeation of Allo across the STRAT-M® membrane was further evaluated by dissolving Allo in the following lead MEs:
(1) ME-A=CAPMUL® MCM:SPAN® 80:TRANSCUTOL® P:water:ethanol, 8:34:34:4:20, by wt %;
(2) ME-B=CAPMUL® MCM:TWEEN® 80:TRANSCUTOL® P:water:ethanol, 13:15:15:42:15, by wt %; and
(3) ME-C=CAPMUL® MCM:TWEEN® 80:SPAN® 80:TRANSCUTOL® P:water:ethanol, 13:8.1:8.1:49.8:6:15, by wt %.

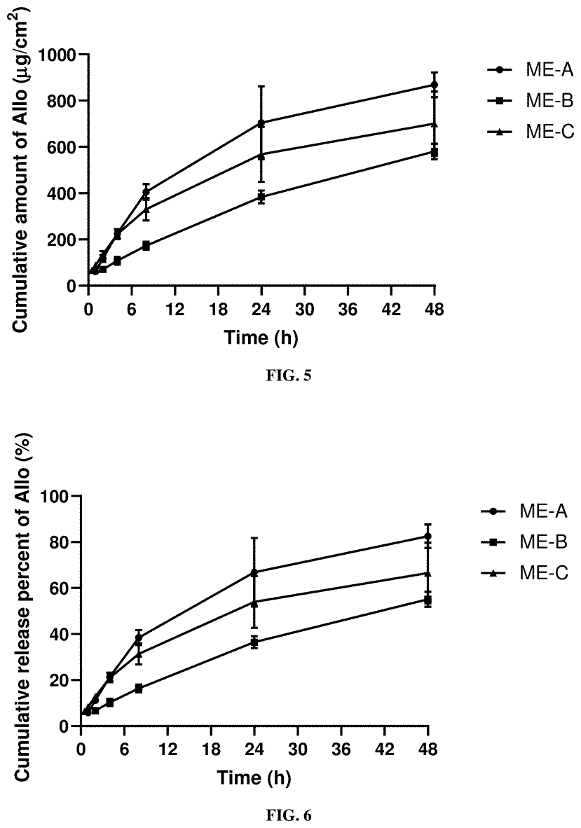

The cumulative amounts of Allo permeated across the membrane for 48 h were 869.13±53.52, 580.09±34.02, and 700.30±138.93 μg/cm² for ME-A, ME-B, and ME-C, respectively (FIG. 5). The percent release of Allo at 48 h was 82.57±5.08, 55.11±3.23, and 66.53±13.20% for ME-A, ME-B, and ME-C, respectively (FIG. 6). The percent release of Allo from ME-A and ME-C was comparable within 4 h. The initial permeations of Allo within 4 h from ME-A and ME-C across the membrane were higher than that from ME-B. The flux of Allo was 54.18±5.52 and 44.44±4.94 μg/cm²/h from ME-A and ME-C, respectively, which was not significantly different, but was higher than that from ME-B (16.88±1.40 μg/cm²/h) (Table 10).

TABLE 10
Flux of Allo from three lead MEs.
Each data point represents mean ± SD (n = 3).

| | | Flux |
|---|---|---|
| | Composition (by wt %) | (μg/cm²/h) |
| ME-A | CAPMUL ® MCM:SPAN ® | 54.18 ± 5.52 |
| | 80:TRANSCUTOL ® P:water:ethanol, | |
| | 8:34:34:4:20 | |
| ME-B | CAPMUL ® MCM:TWEEN ® | 16.88 ± 1.40 |
| | 80:TRANSCUTOL ® P:water:ethanol, | |
| | 13:15:15:42:15 | |
| ME-C | CAPMUL ® MCM:TWEEN ® 80:SPAN ® | 44.44 ± 4.94 |
| | 80:TRANSCUTOL ® P:water:ethanol, | |
| | 13:8.1:8.1:49.8:6:15 | |

### Example 9

### Stability of Allo Formulation

Methods

Quantification of Allopregnanolone (Allo) Using High-Performance Liquid Chromatography (HPLC) Equipped with UV Detector

HPLC-UV system was utilized for the quantification of Allo in samples collected in the long-term stability study. The HPLC system was SHIMADZU LC₂₀₁₀A HT. Chromatographic separation was achieved by using Phenomenex Kinetex Phenyl-hexyl 2.6 μm reverse-phase (150×4.6 mm) column The mobile phase was the mixture of 0.1% acetic acid in water and methanol, 20:80, v/v, and flowed at 0.4 ml/min for 15 min. The injection volume was 10 μl and the UV detection wavelength was 206 nm. A standard curve was constructed at the concentration range of 7.8-500 μg/ml.

Long-Term Stability of Allo Microemulsions (MEs)

The long-term stability of Allo-MEs was determined in the accelerated condition (40° C. and 75±5% relative humidity) for 5 months, and room temperature for additional 1 month (total 6 months). Allo-MEs were prepared in glass vials at the concentrations of 30 and 6 mg/g for ME-A and ME-B, respectively. After the storage conditions and periods, the concentration of Allo in each glass vial was measured using the HPLC-UV system. The physical stability of Allo-MEs was visually evaluated (phase separation, color, transparency, and Allo precipitation). The compositions of Allo-MEs for the long-term stability test were as follows:

(1) ME-A=Capmul® MCM:Span® 80:Transcutol® P:water:ethanol, 8:34:34:4:20, by wt %
(2) ME-B=Capmul® MCM:Tween® 80:Transcutol® P:water:ethanol, 13:15:15:42:15, by wt %

Results

Long-Term Stability of Allo-MEs

Allo was stable in the storage conditions for 6 months. The concentrations of Allo in ME-A and ME-B formulations measured after 6 months were 29.59±0.26 and 6.70±0.14 mg/g (n=3, mean±SD). These concentration changes were within 2 and 12% of nominal concentrations of ME-A (30 mg/g) and ME-B (6 mg/g), respectively (Table 11). There were no physical changes in phase separation, color, and transparency in the storage conditions for 6 months. No precipitation of Allo was observed.

TABLE 11
Long-term stability (6 months) of Allo in MEs
(n = 3, mean ± SD)

| | | Allo concentration, mg/g | | |
|---|---|---|---|---|
| | | (% of nominal concentration) | | |
| ME | Composition (weight %) | Nominal | Day 0 | 6 months |
| ME-A | Capmul ® MCM:Span ® | 30.00 | 30.31 ± 2.98 | 29.59 ± 0.26 |
| | 80:Transcutol ® P:water:ethanol = | | | (98.62 ± 0.87) |
| | 8:34:34:4:20 | | | |
| ME-B | Capmul ® MCM:Tween ® | 6.00 | 6.11 ± 0.03 | 6.70 ± 0.14 |
| | 80:Transcutol ® P:water:ethanol = | | | (111.61 ± 2.40) |
| | 13:15:15:42:15 | | | |

### Example 10

### Stability and Solubility of Allo Formulations

Based on a ternary phase diagram presented (FIG. 1B), weight percent of each inactive ingredient with higher percent of water phase was determined. Forty percent (40%) of I-10CD was dissolved in distilled water (DW), which was used for the water phase of microemulsions. Capmul® MCM C₈ was used instead of Capmul® MCM.

Methods

The procedure for preparing the formulations were as follows:

1. Allo was measured and added to a glass vial.

2. Capmul® MCM (or Capmul® MCM C₈) and surfactant/co-surfactant were added into the vial.

3. The contents of the vial were vortexed for 1-2 min.

4. Water or water containing cyclodextrin (Dexolve or I-1(3CD) was added into the vial, and then ethanol was added into the vial.

5. The contents of the vial were vortexed for 1-2 mins until All was clearly dissolved.

6. Clear and single-phase Allo microemulsions were formed.

7. The Allow microemulsions were stored at refrigerator temperature (4° C.) or at room temperature.

Results

TABLE 12
Stability (room temperature)

| | | Allo concentration | |
|---|---|---|---|
| | | (mg/g) | |
| ME | Composition (weight %) | Day 0 | 1 week |
| ME-B-HβCD-1 | Capmul ® MCM | 4.57 | 5.01 ± 0.32 |
| | C8:Tween ® 80:Transcutol ® | | |
| | P:40%^{a} HβCD-DW^{b} = | | |
| | 1:7.5:7.5:85^{c} | | |

^{a}40% = weight of HβCD (g) in 100 mL of DW
^{b}DW = distilled water
cBy wt%

TABLE 13
Solubility

| ME | Composition (weight %) | Solubility (mg/mL) |
|---|---|---|
| ME-B-HβCD-1 | Capmul ® MCM | 5.0 |
| | C8:Tween ® 80:Transcutol ® | |
| | P:40%^{a} HβCD-DW^{b} = | |
| | 1:7.5:7.5:85 | |
| ME-B-HβCD-2 | Capmul ® MCM | 5.0 |
| | C8:Tween ® 80:Transcutol ® | |
| | P:40%^{a} HβCD-DW^{b}:ethanol = | |
| | 1:7.5:7.5:83:2 | |
| ME-B-HβCD-3 | Capmul ® MCM | 5.0 |
| | C8:Tween ® 80:Transcutol ® | |
| | P:40%^{a} HβCD-DW^{b}:ethanol = | |
| | 1:7.5:7.5:80:5 | |

^{a}40% = weight of HβCD (g) in 100 mL of DW
^{b}DW = distilled water
^{c}By wt%

We claim:

1. A formulation for treating or preventing neuronal damage and/or the associated cognitive decline or impairment, caused by Alzheimer's disease and/or other neurodegenerative diseases, neurological injury, or age-related neuronal decline, comprising
a therapeutic agent, selected from the group consisting of 3a-hydroxy-5a-pregnan-20-one, a derivative or analogue thereof, and a pharmaceutically acceptable salt of the derivative or analogue; and
a pharmaceutically acceptable carrier comprising water, one or more lipophilic compounds, a solubilizer, a surfactant, and optionally a co-surfactant, wherein the carrier forms a stable microemulsion,
wherein the therapeutic agent is dissolved in the carrier, and
wherein the weight percent of the surfactant or the combination of the surfactant and the co-surfactant relative to the carrier is between about 10% and about 90%.

2. The formulation of claim 1, wherein the therapeutic agent is 3a-hydroxy-5a-pregnan-20-one.

3. The formulation of claim 1 , wherein the solubility of the therapeutic agent in the carrier is higher than the solubility of the therapeutic agentin a corresponding carrier without the one or more lipophilic compounds, surfactant, or co-surfactant.

4. The formulation of claim 1, wherein the one or more lipophilic compounds are selected from fatty acids, fatty acid esters, and combinations thereof.

5. The formulation of claim 1, wherein the one or more lipophilic compounds are selected from C₆-C₁₂ medium-chain, saturated or non-saturated, mono-, di- or tri-glycerides.

6. The formulation of claim 1, wherein the one or more lipophilic compounds are selected from the group consisting of caprylic monoglyceride, caprylic diglyceride, capric monoglyceride, capric diglyceride, and combinations thereof.

7. The formulation of claim 1, wherein the carrier comprises an oil, wherein the one or more lipophilic compounds originally belonged to the oil.

8. The formulation of claim 7, wherein the oil is a mixture containing caprylic/capric mono- and diglycerides.

9. The formulation of claim 1, wherein the surfactant is a non-ionic surfactant.

10. The formulation of claim 1, wherein the surfactant is selected from the group consisting of polysorbates, sorbitan alkanoates, polyoxyethylene fatty acid esters, and combinations thereof.

11. The formulation of claim 1, wherein the surfactant is sorbitan monooleate, Polysorbate 80, or a combination thereof, optionally at a weight ratio of about 1.

12. The formulation of claim 1, wherein the co-surfactant is diethylene glycol monoethyl ether.

13. The formulation of claim 1, wherein the carrier further comprises a transdermal penetration enhancer.

14. The formulation of claim 13, wherein the transdermal penetration enhancer is ethanol, propylene glycol, or glycerol.

15. The formulation of claim 1, wherein the microemulsion is stable at 40° C. and 75% relative humidity for at least a month without precipitation of the therapeutic agent, color change of the formulation, or transparency change of the formulation.

16. The formulation of claim 1, wherein the one or more lipophilic compounds, surfactant, co-surfactant, and/or transdermal penetration enhancer meets the requirements of the U.S. Food and Drug Administration as generally recognized as safe compounds.

17. The formulation of claim 1, wherein
(1) the concentration of the therapeutic agent in the formulation is between about 0.5 and about 100mg/ml;
(2) the weight percent of the one or more lipophilic compounds or the oil relative to the carrier is more than 0.01% and up to 30%,
(3) the weight percent of the surfactant or the combination of the surfactant and the co-surfactant relative to the carrier is between about 10% and about 90%;
(4) the weight percent of the transdermal penetration enhancer relative to the carrier is up to about 20%; and/or
(5) the weight percent of water relative to the carrier is up to about

88.

18. The formulation of claim 1, wherein the carrier comprises water, the one or more lipophilic compounds, the surfactant, the co-surfactant, and the tissue penetration enhancer.

19. The formulation of claim 18, wherein
(1) the therapeutic agent is 3a-hydroxy-5a-pregnan-20-one;
(2) the one or more lipophilic compounds are selected from the group consisting of caprylic monoglyceride, caprylic diglyceride, capric monoglyceride, capric diglyceride, and combinations thereof;
(3) the surfactant is sorbitan monooleate, Polysorbate 80, or a combination of sorbitan monooleate and Polysorbate 80 at a weight ratio of about 1;
(4) the co-surfactant is diethylene glycol monoethyl ether; and
(5) the transdermal penetration enhancer is ethanol.

20. A dosage unit kit of the formulation of claim 1, comprising one or more containers for dry components and one or more containers for liquid components, which are mixed together to form the formulation before administration to a subject in need thereof.

21. A method for treating or preventing neuronal damage and/or the associated cognitive decline or impairment, caused by Alzheimer's disease and/or other neurodegenerative diseases, comprising administering an effective amount of the formulation of claim 1.

22. The method of claim 21, wherein the formulation is administered topically to a mucosal surface or the skin.

23. The method of claim 21, wherein the formulation is administered using a delivery vehicle selected from the group consisting of microneedles, intranasal sprays, buccal or sublingual films, transdermal patches capsules and sprays.

24. The method of claim 21, wherein the formulation is administered using a microneedle device.

25. A microneedle device comprising allopregnanolone or a derivative or salt thereof.

26. The formulation of claim 1, wherein the weight percent of the solubilizer relative to the carrier is between about 10% and about 90%.

27. The formulation of claim 1, wherein the 3a-hydroxy-5a-pregnan-20-one is dissolved in the solubilizers within the carriers, optionally wherein a predominant quantity of the 3a-hydroxy-5a-pregnan-20-one is dissolved in the solubilizers.

28. The formulation of claim 1, wherein the solubilizers comprise polysaccharides; water-soluble organic solvents; non-ionic surfactants that can enhance hydroxy-5a-pregnan-20-one solubility; water-insoluble lipids;
organic liquids/semi-solids; phospholipids; or combinations thereof.

29. The formulation of claim 1, wherein the solubilizers comprise polysaccharides comprising cyclodextrins, derivatives of cyclodextrins, chemically modified cellulose, or combinations thereof.

30. The formulation of claim 29, wherein the cyclodextrins or derivatives thereof, are selected from the group consisting of α-cyclodextrins, β-cyclodextrins, and γ-cyclodextrins.

31. The formulation of claim 30, wherein the α-cyclodextrins are selected from the group consisting of hydroxypropyl-β-cyclodextrins, sulfobutylether-β-cyclodextrins, heptakis-6-sulfoethylsulfanyl-6-deoxy-β-cyclodextrin, heptakis-6-methylsulfanyl-6-deoxy-2-(2-(2-(2-methoxyethoxy)ethoxy)ethyl)]-β-cyclodextrins, heptakis-6-thioglyceryl-6-deoxy-β-cyclodextrins, and combinations thereof.

32. The formulation of claim 1, wherein the solubilizer and the therapeutic agent form a host-guest complex.

33. The formulation of claim 1, wherein the formulation increases or retains hippocampal volume during and/or after administration, as measured by magnetic resonance imaging.
